(19) United States
(12) Patent Application Publication
(10) Pub. No.: US 2012/0065210 A1
(43) Pub. Date: Mar. 15, 2012

(54) SUBSTITUTED HEXAHYDROPYRROLO[1,2-C]IMIDAZOLONES

(76) Inventors: Xin-Jie Chu, Livingston, NJ (US); Qingjie Ding, Bridgewater, NJ (US); Nan Jiang, Pine Brook, NJ (US); Jin-Jun Liu, Warren Township, NJ (US); Tina Morgan Ross, Royersford, PA (US); Zhuming Zhang, Hillsborough, NJ (US)

(21) Appl. No.: 13/226,523

(22) Filed: Sep. 7, 2011

Related U.S. Application Data
(60) Provisional application No. 61/382,969, filed on Sep. 15, 2010.

(51) Int. Cl. A61K 31/4188; A61P 35/00; A61K 31/454; A61K 31/4985; C07D 487/04; A61K 31/4439

(52) U.S. Cl. 514/249; 548/302.7; 546/273.1; 546/199; 544/349; 514/387; 514/338; 514/322

(57) ABSTRACT

There are provided compounds of formula I

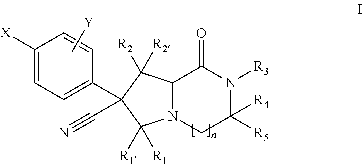

or a pharmaceutically acceptable salt thereof,
wherein X, Y, R₁, R_{1′}, R₂, R_{2′}, R₃, R₄, R₅ are as defined herein. The compounds exhibit activity as anticancer agents.

### PRIORITY TO RELATED APPLICATION(S)

This application claims the benefit of U.S. Provisional Application No. 61/382,969, filed Sep. 15, 2010, which is hereby incorporated by reference in its entirety.

### FIELD OF THE INVENTION

The present invention relates to hexahydro-pyrrolo[1,2-c]imidazolones and octahydro-pyrrolo[1,2-a]pyrazine-7-carbonitriles which act as inhibitors of MDM2-p53 interactions and are useful in the amelioration or treatment of cancer, especially solid tumors.

### BACKGROUND OF THE INVENTION

p53 is a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor which is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This feedback control loop insures that both MDM2 and p53 are kept at a low level in normal proliferating cells. MDM2 is also a cofactor for E2F, which plays a central role in cell cycle regulation.

The ratio of MDM2 to p53 (E2F) is dysregulated in many cancers. Frequently occurring molecular defects in the p16INK4/p19ARF locus, for instance, have been shown to affect MDM2 protein degradation. Inhibition of MDM2-p53 interaction in tumor cells with wild-type p53 should lead to accumulation of p53, cell cycle arrest and/or apoptosis. MDM2 antagonists, therefore, can offer a novel approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as p53 and activates E2F-dependent transcription of cyclin A, suggesting that MDM2 antagonists might also have effects in p53 mutant cells.

### SUMMARY OF THE INVENTION

One aspect of the invention is a compound of formula I

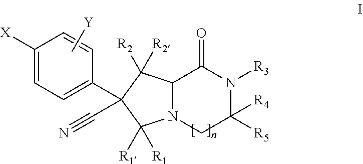

or a pharmaceutically acceptable salt thereof, wherein X, Y, R₁, R_{1′}, R₂, R_{2′}, R₃, R₄, R₅ are as defined below.

The present invention also relates to pharmaceutical compositions comprising one or more compounds of the invention, or a pharmaceutically acceptable salt, and a pharmaceutically acceptable carrier or excipient.

The present invention further relates to a method of treating, ameliorating or preventing cancer in a mammal, preferably a human, comprising administering to said mammal a therapeutically effective amount of a compound according to the invention or a pharmaceutically acceptable salt thereof.

### DETAILED DESCRIPTION OF THE INVENTION

### Definitions

As used herein, the following terms shall have the following definitions.

The term “alkyl” refers to straight- or branched-chain saturated hydrocarbon groups having from 1 to about 12 carbon atoms, including groups having from 1 to about 7 carbon atoms. In certain embodiments, alkyl substituents may be lower alkyl substituents. The term “lower alkyl” refers to alkyl groups having from 1 to 6 carbon atoms, preferably from 1 to 4 carbon atoms. Examples of alkyl groups include, but are not limited to, methyl, ethyl, n-propyl, i-propyl, n-butyl, s-butyl, t-butyl, n-pentyl, and s-pentyl.

The term “alkenyl” as used herein means an unsaturated straight-chain or branched aliphatic hydrocarbon group containing at least one double bond and having 2 to 6, preferably 2 to 4 carbon atoms. Examples of such “alkenyl group” are vinyl, ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

“Alkoxy, alkoxyl or lower alkoxy” refers to any of the above lower alkyl groups which is attached to the remainder of the molecule by an oxygen atom (RO—). Typical lower alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

Amino means the group —NH₂.

“Aryl” means a monovalent, monocyclic or bicyclic, aromatic carboxylic hydrocarbon radical, preferably a 6-10 member aromatic ring system. Preferred aryl groups include, but are not limited to, phenyl, naphthyl, tolyl, and xylyl.

Carboxyl or carboxy means the monovalent group —COOH. Carboxy lower alkyl means —COOR, wherein R is lower alkyl. Carboxy lower alkoxy means —COOROH wherein the R is lower alkyl.

Carbonyl means the group

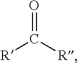

where R′ and R″ independently can be any of a number of chemical groups including alkyl.

The term “cycloalkyl” as used herein means any stable monocyclic or polycyclic system which consists of carbon atoms only, any ring of which being saturated, and the term “cycloalkenyl” is intended to refer to any stable monocyclic or polycyclic system which consists of carbon atoms only, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds. Examples of cycloalkenyls include, but are not limited to, cyclopentenyl or cyclohexenyl.

The term “halogen” as used herein means fluorine, chlorine, bromine, or iodine, preferably fluorine and chlorine.

“Heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazole substituted or unsubstituted triazolyl and substituted or unsubstituted tetrazolyl.

In the case of aryl or heteroaryl which are bicyclic it should be understood that one ring may be aryl while the other is heteroaryl and both being substituted or unsubstituted.

“Hetero atom” means an atom selected from N, O and S.

“Heterocycle” or “heterocyclic ring” means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. Examples include pyrrolidin-2-yl; pyrrolidin-3-yl; piperidinyl; morpholin-4-yl and the like which in turn can be substituted.

Hydroxy or hydroxyl is a prefix indicating the presence of a monovalent —O—H group.

“IC₅₀” refers to the concentration of a particular compound required to inhibit 50% of a specific measured activity. IC₅₀ can be measured, inter alia, as is described subsequently in Example 72.

“Lower” as in “lower alkenyl” means a group having 1 to 6 carbon atoms.

“Nitro” means —NO₂.

Oxo means the group ═O.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoro acetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (1995) at pgs. 456-457.

“Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent. In the specification where indicated the various groups may be substituted by preferably, 1-3 substituents independently selected from the group consisting of H, carboxyl, amido, hydroxyl, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle.

In one embodiment, the present invention relates to compounds of formula I

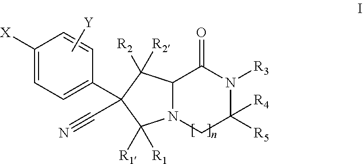

### Wherein

X is selected from the group consisting of H, F, Cl, Br, I, cyano, nitro, lower alkyl, lower alkynyl and lower alkoxy;

### Y is H or F;

n is selected from 0, 1 or 2;
R₁ and R_{1′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₂ and R_{2′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R₄ and R₅ are independently selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or they can together form an oxo group
and a pharmaceutically acceptable salt or ester thereof.

Preferred are compounds of formula II

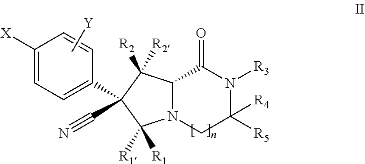

wherein
X is selected from the group consisting of H, F, Cl, Br, I, cyano, nitro, lower alkyl, lower alkynyl and lower alkoxy;

### Y is H or F;

n is selected from 0, 1 or 2;
R₁ and R_{1′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₂ and R_{2′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R₄ and R₅ are independently selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or they can together form an oxo group
and the enantiomers thereof or a pharmaceutically acceptable salt or ester thereof.

More preferred are compounds of formula II in which R_{2′} is H, R₂ is selected from the group consisting of a substituted phenyl as shown in formula IIa:

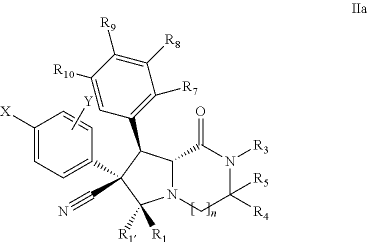

wherein,
X is selected from the group consisting of H, F, Cl and Br, I, cyano, nitro, lower alkyl, lower alkynyl and lower alkoxy;

### Y is H or F;

n is selected from 0, 1 or 2;
R₈ is selected from the group consisting of F, Cl and Br;
R₇, R₉ and R₁₀ are H or F with the proviso that at least two of R₇, R₉ and R₁₀ are hydrogen;
R₁ and R_{1′} are independently selected from the group consisting of hydrogen, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R₄ and R₅ are independently selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or they can together form an oxo group
and the enantiomers thereof or a pharmaceutically acceptable salt or ester thereof.

Further preferred are compounds of formula III in which R_{1′} is hydrogen, R₁ is selected from a group consisted of substituted lower alkyl shown as in formula III:

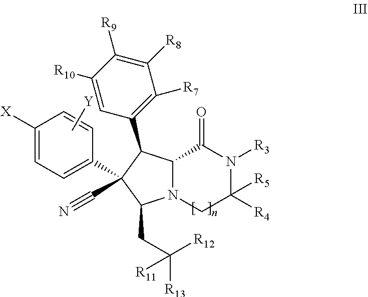

### Wherein,

X is selected from the group consisting of H, F, Cl and Br, I, cyano, nitro, lower alkyl, lower alkynyl and lower alkoxy;

### Y is H or F;

n=0, 1 or 2;
R₈ is selected from the group consisting of F, Cl and Br;
R₇, R₉, R₁₀ are selected from H or F with the proviso that at least two of R₇, R₉ and R₁₀ are hydrogen;
R₁₁, R₁₂ are both methyl, or linked to form a cyclopropyl, cyclobutyl, cyclopentyl or cyclohexyl group;
R₁₃ is (CH₂)ₘ—R₁₄;
m is selected from 0, 1 or 2;
R₁₄ is selected from hydrogen, hydroxyl, lower alkyl, lower alkoxy, lower cycloalkenyl, substituted cycloalkenyl, lower cycloalkyl, substituted cycloalkyl, aryl, substituted aryl, hetereoaryl, substituted heteroaryl, hetereocycle or substituted heterocycle;
R₃ is aryl, substituted aryl, heteroaryl or substituted heteroaryl;
R₄ and R₅ are independently selected from H, lower alkyl, substituted lower alkyl, lower cycloalkyl, substituted lower cycloalkyl, lower alkenyl, substituted lower alkenyl, lower cycloalkenyl or substituted lower cycloalkenyl or they can together form an oxo group and enantiomers thereof or a pharmaceutically acceptable salt or ester thereof

Further preferred are compounds of formula III in which R₁₁, R₁₂, R₁₃ are methyl as shown in formula IV,

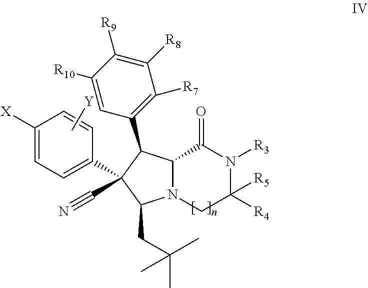

wherein,
X is selected from the group consisting of H, F, Cl and Br, I, cyano, nitro, lower alkyl, lower alkynyl and lower alkoxy;

### Y is H or F;

n=0, 1 or 2;
R₈ is selected from the group consisting of F, Cl and Br;
R₇, R₉, R₁₀ is selected from H or F with the proviso that at least two of R₇, R₉, R₁₀ are hydrogen;
R₃ is selected from the group consisting aryl, substituted aryl, heteroaryl or substituted heteroaryl wherein the substituents are selected from H, carboxyl, amido, hydroxyl, cyano, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle;
R₄ and R₅ are independently selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or they can together form an oxo group
and the enantiomers thereof or a pharmaceutically acceptable salt or ester thereof.

Further preferred are compounds of formula IV in which n=0, R₅=H as shown in formula V.

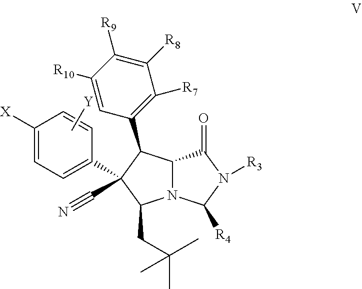

wherein,
X is selected from the group consisting of H, F, Cl and Br, I, cyano, nitro, lower alkyl, lower alkynyl and lower alkoxy;
Y is a mono substituting group consisting of H or F;
R₈ is selected from the group consisting of F, Cl and Br;
R₇, R₉, R₁₀ is selected from H or F with the proviso that at least two of R₇, R₉, R₁₀ are hydrogen;
R₃ is selected from the group consisting aryl, substituted aryl, heteroaryl or substituted heteroaryl wherein the substituents are selected from H, carboxyl, amido, hydroxyl, cyano, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle;
R₄ is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl;
and the enantiomers thereof and a pharmaceutically acceptable salt or ester thereof.

Further preferred are compounds of formula IV where R₄ and R₅ are both hydrogen and n=1 as in formula VI.

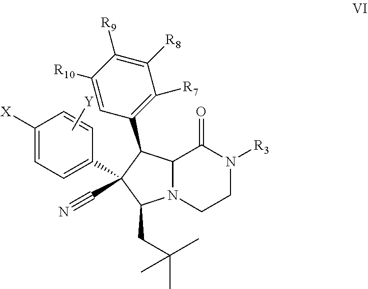

wherein,
X is selected from the group consisting of H, F, Cl, Br, I, cyano, nitro, lower alkyl, lower aklynyl and lower alkoxy; Y is H or F;
R₈ is selected from the group consisting of F, Cl and Br;
R₇, R₉, R₁₀ is selected from H or F with the proviso that at least two of R₇, R₉, R₁₀ are hydrogen;
R₃ is selected from the group consisting aryl, substituted aryl, heteroaryl or substituted heteroaryl wherein the substituents are selected from H, carboxyl, amido, hydroxyl, cyano, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle and the enantiomers thereof and a pharmaceutically acceptable salt or ester thereof.

Compounds prepared according to the invention include:
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-methoxy-benzoic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-benzoic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-methoxy-benzoic acid;
[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-acetic acid;
2-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-N,N-bis-(2-methoxy-ethyl)-acetamide;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-hydroxy-benzoic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-trifluoromethyl-benzoic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-fluoro-benzoic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-trifluoromethoxy-benzoic acid;
(5S,6R,7S,7aR)-7-(3-chloro-2-fluoro-phenyl)-6-(4-chloro-2-fluoro-phenyl)-5-(2,2-dimethyl-propyl)-2-(3-hydroxy-phenyl)-1-oxo-hexahydro-pyrrol o[1,2-c]imidazole-6-carbonitrile;
(5S,6R,7S,7aR)-7-(3-chloro-2-fluoro-phenyl)-6-(4-chloro-2-fluoro-phenyl)-5-(2,2-dimethyl-propyl)-2-(2-hydroxy-phenyl)-1-oxo-hexahydro-pyrrolo[1,2-c]imidazole-6-carbonitrile;
(5S,6R,7S,7aR)-7-(3-chloro-2-fluoro-phenyl)-6-(4-chloro-2-fluoro-phenyl)-5-(2,2-dimethyl-propyl)-2-(2-fluoro-4-methoxy-phenyl)-1-oxo-hexahydro-pyrrolo[1,2c]imidazole-6-carbonitrile;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzoic acid methyl ester;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzoic acid methyl ester;
4-[(5S,6R,7S,7aR)-7-(3-chloro-2-fluoro-phenyl)-6-(4-chloro-2 fluoro-phenyl)-6-cyano-5-(2,2-dimethyl-propyl)-1-oxo-tetrahydro-pyrrolo[1,2-c]imidazol-2-yl]-3-methoxy-benzamide;
4-[(5S,6R,7S,7aR)-7-(3-chloro-2-fluoro-phenyl)-6-(4-chloro)-2-fluoro-phenyl)-6-cyano-5-(2,2-dimethyl-propyl)-1-oxo-tetrahydro-pyrrolo[1,2-c]imidazol-2-yl]-3-methoxy-benzoic acid methyl ester;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzamide;
5-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-pyridine-2-carboxylic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzoic acid;
(5S,6R,7S,7aR)-7-(3-chloro-2-fluoro-phenyl)-6-(4-chloro-2-fluoro-phenyl)-5-(2,2-dimethyl-propyl)-2-(1-methanesulfonyl-piperidin-4-yl)-1-oxo-hexahydro-pyrrolo[1,2-c]imidazole-6-carbonitrile;
(5S,6R,7S,7aR)-2-(1-acetyl-piperidin-4-yl)-7-(3-chloro-2-fluoro-phenyl)-6-(4-chloro-2-fluoro-phenyl)-5-(2,2-dimethyl-propyl)-1-oxo-hexahydro-pyrrolo[1,2-c]imidazole-6-carbonitrile;
rac-(5S,6R,7R,7aR)-7-(3-chloro-phenyl)-6-(4-chlorophenyl)-5-(2,2-dimethyl-propyl)-2-methyl-1,3-dioxo-hexahydro-pyrrolo[1,2-c]imidazole-6-carbonitrile;
rac-(5S,6R,7R,7aR)-7-(3-chloro-phenyl)-6-(4-chlorophenyl)-5-(2,2-dimethyl-propyl)-2-ethyl-1,3-dioxo-hexahydro-pyrrolo[1,2-c]imidazole-6-carbonitrile;
rac-(5R,6R,7R,7aR)-7-(3-chloro-phenyl)-6-(4-chlorophenyl)-5-(2,2-dimethyl-propyl)-2-ethyl-1,3-dioxo-hexahydro-pyrrolo[1,2-c]imidazole-6-carbonitrile;
rac-3-[(5S,6R,7R,7aR)-7-(3-chloro-phenyl)-6-(4-chlorophenyl)-6-cyano-5-(2,2-dimethyl-propyl)-1,3-dioxo-tetrahydro-pyrrolo[1,2-c]imidazole-2-yl]-propionic acid ethyl ester;
rac-(2R,3R,4R,5S)-3-(3-chloro-phenyl)-4-(4-chloro-phenyl)-4-cyano-5-(2,2-dimethyl-propyl)-1-[(4-hydroxy-butylcarbamoyl)-methyl]-pyrrolidine-2-carboxylic acid tert-butyl ester;
rac-(5S,6R,7R,7aR)-7-(3-chloro-phenyl)-6-(4-chloro-phenyl)-5-(2,2-dimethyl-propyl)-2-(4-hydroxy-butyl)-1,3-dioxo-hexahydro-pyrrolo[1,2-c]imidazole-6-carbonitrile;
rac-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid methyl ester;
rac-4-[(5S,6R,7S,7aR)-7-(3-chloro-2-fluoro-phenyl)-6-(4-chloro-2-fluoro-phenyl)-2-(4-cyano-phenyl)-5-(2,2-dimethyl-propyl)-1,3-dioxo-hexahydro-pyrrolo[1,2-c]imidazole-6-carbonitrile;
rac-4-[(5S,6R,7S,7aR)-7-(3-chloro-2-fluoro-phenyl)-6-(4-chloro-2-fluoro-phenyl)-5-(2,2-dimethyl-propyl)-1,3-dioxo-2-phenyl-hexahydro-pyrrolo[1,2-c]imidazole-6-carbonitrile;
rac-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid;
chiral-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid;
rac-3-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid ethyl ester;
rac-3-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid;
chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-benzoic acid;
rac-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-benzoic acid;
rac-4-[(3S,5S,6R,7S,7aR)-7-(3-chloro-2-fluoro-phenyl)-6-(4-chloro-2-fluoro-phenyl)-6-cyano-3-cyclohexylmethyl-5-(2,2-dimethyl-propyl)-1-oxo-tetrahydro-pyrrolo[1,2-c]imidazol-2-yl]-benzoic acid;
chiral 4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-benzoic acid;
chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-2-methoxy-benzoic acid;
rac-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-3-methoxy-benzoic acid;
rac 4-[(3S,5S,6R,7S,7aR)-3-butyl-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2c]imidazol-2-yl]-2-methoxy-benzoic acid;
4-[(3S,5S,6R,7S,7aR)-3-but-3-enyl-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-methoxy-benzoic acid;
rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-3-methoxy-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-methyl-1-oxo-tetrahydro-pyrrolo[1,2-c]imidazol-2-yl]-3-methoxy-benzoic acid;
rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isobutyl-1-oxo-tetrahydro-pyrrolo[1,2-c]imidazol-2-yl]-3-methoxy-benzoic acid;
chiral-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-3-methoxy-benzoic acid
chiral-4-[(3S,5S,6R,7S,7aR)-7-(3-chloro-2-fluoro-phenyl)-6-(4-chloro-2-fluoro-phenyl)-6-cyano-3-cyclohexylmethyl-5-(2,2-dimethyl-propyl)-1-oxo-tetrahydro-pyrrolo[1,2-c]imidazol-2-yl]-3-methoxy-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-3-methoxy-benzoic acid;
chiral-4-[(3S,5S,6R,7S,7aR)-7-(3-chloro-2-fluoro-phenyl)-6-(4-chloro-2-fluoro-phenyl)-6-cyano-3-cyclobutyl-5-(2,2-dimethyl-propyl)-1-oxo-tetrahydro-pyrrolo[1,2-c]imidazol-2-yl]-3-methoxy-benzoic acid;
rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-hydroxymethyl-1-oxo-tetrahydro-pyrrolo[1,2-c]imidazol-2-yl]-benzoic acid;
rac 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-ethyl-1-oxo-tetrahydro-pyrrolo[1,2c]imidazol-2-yl]-2-methoxy-benzoic acid;
rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-3-methoxy-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-hydroxymethyl-1-oxo-tetrahydro-pyrrolo[1,2-c]imidazol-2-yl]-3-methoxy-benzoic acid;
rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-1-oxo-3-(tetrahydro-pyran-4-ylmethyl)-tetrahydro-pyrrolo[1,2-c]imidazol-2-yl]-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-1-oxo-3-(tetrahydro-pyran-4-ylmethyl)-tetrahydro-pyrrolo[1,2-c]imidazol-2-yl]-2-methoxy-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-2-methoxy-benzoic acid;
rac-(6S,7R,8S,8aR)-8-(3-chloro-2-fluoro-phenyl)-7-(4-chloro-2-fluoro-phenyl)-2-((S)-3,4-dihydroxy-butyl)-6-(2,2-dimethyl-propyl)-1-oxo-octahydro-pyrrolo[1,2-a]pyrazine-7-carbonitrile and
chiral (6S,7R,8S,8aR)-8-(3-chloro-2-fluoro-phenyl)-7-(4-chloro-2-fluoro-phenyl)-2-((S)-3,4-dihydroxy-butyl)-6-(2,2-dimethyl-propyl)-1-oxo-octahydro-pyrrolo[1,2-a]pyrazine-7-carbonitrile.

Compounds disclosed herein and covered by formula I above may exhibit tautomerism or structural isomerism. It is intended that the invention encompasses any tautomeric or structural isomeric form of these compounds, or mixtures of such forms, and is not limited to any one tautomeric or structural isomeric form depicted in the formulas above.

### Dosages

The compounds of the present invention are inhibitors of MDM2-p53 interactions and are thus useful in the treatment or control of cell proliferative disorders, in particular chemoprevention of cancer. Chemoprevention is defined as inhibiting the development of invasive cancer by either blocking the initiating mutagenic event or by blocking the progression of pre-malignant cells that have already suffered an insult of inhibiting tumor relapse. These compounds and formulations containing said compounds are anticipated to be particularly useful in the treatment or control of solid tumors, such as, for example, breast, colon, lung and prostate tumors.

A “therapeutically effective amount” or “effective amount” of a compound in accordance with this invention means an amount of compound that is effective to prevent, alleviate or ameliorate symptoms of disease or prolong the survival of the subject being treated.

The therapeutically effective amount or dosage of a compound according to this invention can vary within wide limits. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration; it may be given as continuous infusion.

### Compositions/Formulations

In an alternative embodiment, the present invention includes pharmaceutical compositions comprising at least one compound of formula I, or a pharmaceutically acceptable salt thereof and a pharmaceutically acceptable excipient and/or carrier.

These pharmaceutical compositions can be suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a formula I compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

The pharmaceutical preparations of the invention can also contain preserving agents, solubilizing agents, stabilizing agents, wetting agents, emulsifying agents, sweetening agents, coloring agents, flavoring agents, salts for varying the osmotic pressure, buffers, coating agents or antioxidants. They can also contain other therapeutically valuable substances, including additional active ingredients other than those of formula I.

General Synthesis of Substituted hexahydropyrrolo[1,2-c]imidazolones and octahydro-pyrrolo[1,2-a]pyrazine-7-carbonitriles

The present invention provides methods for the synthesis of the substituted hexahydropyrrolo[1,2-c]imidazolones and octahydro-pyrrolo[1,2-a]pyrazine-7-carbonitriles.

The compounds of the invention can be prepared by processes known in the art. Suitable processes for synthesizing these compounds are also provided in the examples. Generally, compounds of formula I can be synthesized according to one of the below described synthetic routes.

The key transformations are a convergent [2+3] cycloaddition of imine A and activated olefin B to generate pyrrolidine-3-carbonitrile compounds C in a stereoselective manner. Compound C then can be used directly to make amide D or resolved first and then used to make chiral amide D. Compound D was then reacted with aldehyde or a suitable alkylation reagent to generate the desired target I.

The starting materials are either commercially available or can be synthesized by methods known to those of ordinary skill in the art. Preparations of intermediates A and B are illustrated in Schemes 1 and 2 below. In general an appropriately selected aldehyde or ketone can be reacted with glycine tert-butyl ester or glycine methyl ester to generate imine A as a crude product (see Scheme 1 below).

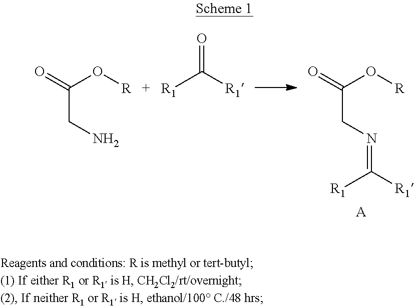

An intermediate of formula B can be made from a base-catalyzed condensation reaction of appropriately selected substituted-phenyl acetonitriles and aldehydes. The reaction proceeds in a highly stereoselective manner with the Z-isomer as the major or exclusive product (see scheme 2 below).

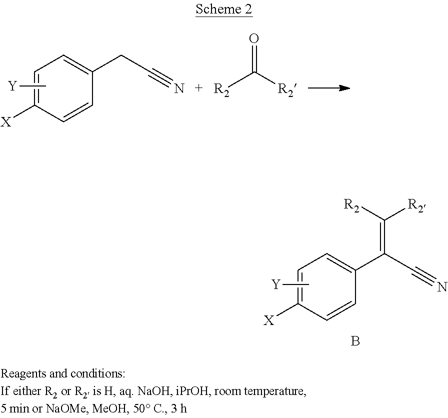

As is illustrated in Scheme 3 below, pyrrolidines of formula C can be made from intermediates A and B by a convergent 1,3-dipolar cycloaddition reaction mediated by lewis acid

AgF and triethylamine, followed by hydrolysis. The [2+3] cycloaddition reactions of azomethine ylides 1,3-dipoles (that were generated from reacting intermediate A with AgF) with olefinic dipolarphiles of formula B to form pyrrolidine ring are described in the literature, including Jorgensen, K. A. et al (Org. Lett. 2005, Vol 7, No. 21, 4569-4572), Grigg, R. et al (Tetrahedron, 1992, Vol 48, No. 47, 10431-10442; Tetrahedron, 2002, Vol 58, 1719-1737), Schreiber, S. L. et al (J. Am. Chem. Soc., 2003, 125, 10174-10175), and Carretero, J. C. et al (Tetrahedron, 2007, 63, 6587-6602).

Compounds of formula C are subsequently converted to compounds of formula D by amide formation with various amines using HATU as the coupling reagent. Other known arts using different activating agents like EDCI, HOBT or oxylyl chloride also work.

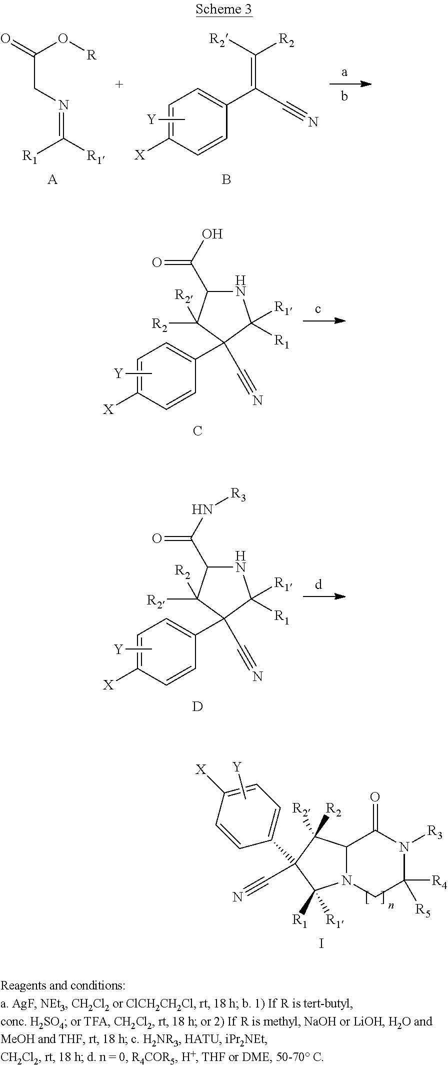

Racemic C can be readily resolved into two optically pure or enriched chiral enantiomers C1 and C2 by separation using chiral Super Fluid Chromatography (SFC). (see Scheme 4 below). Racemic D can be resolved by a similar manner (see Scheme 5 below) and formular I can also be resolved by the similar method.

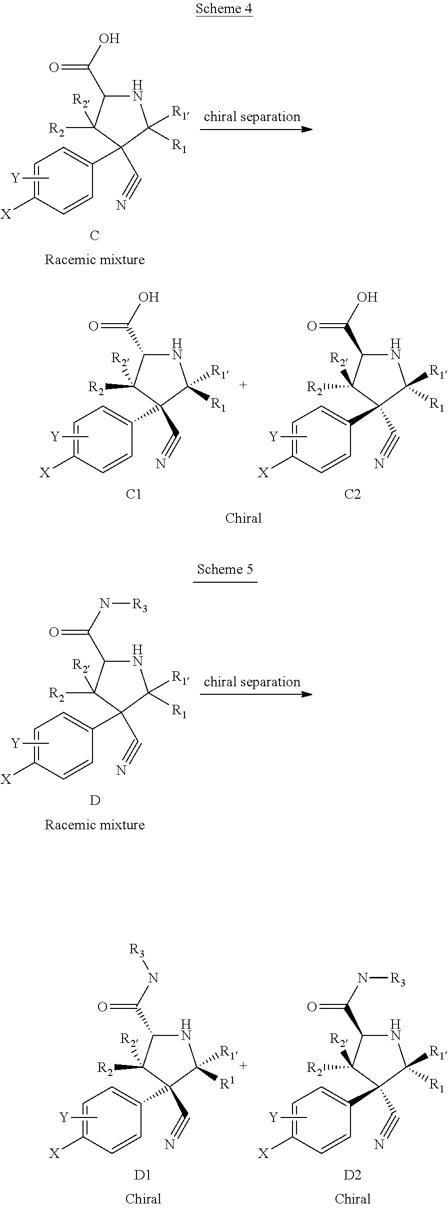

Resolution methods are well known, and are summarized in “Enantiomers, Racemates, and Resolutions” (Jacques, J. et. al. John Wiley and Sons, NY, 1981). Methods for chiral HPLC are also well known, and are summarized in

“Separation of Enantiomers by Liquid Chromatographic Methods” (Rirkle, W. H. and Finn, J in Asymmetric Synthesis' Vol. 1, Morrison, J. D., Ed. Academic Press, In., NY 1983, pp. 87-124).

When n=0 and R₄ and R₅ are together to form an oxo group, compounds of Formular I can generally be prepared as described in Scheme 6.

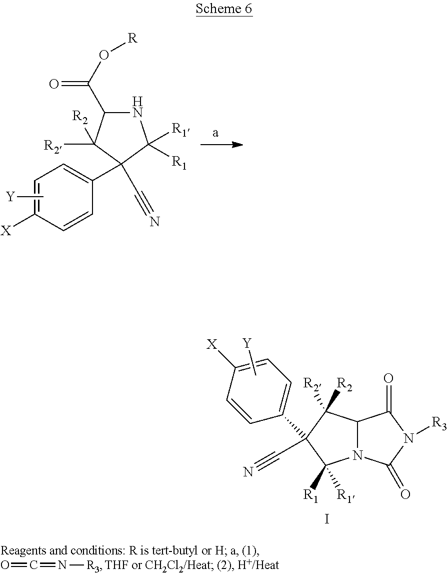

When n=1 and R₄ and R₅ are both hydrogen, compounds of Formular I can generally be prepared as described in Scheme 7.

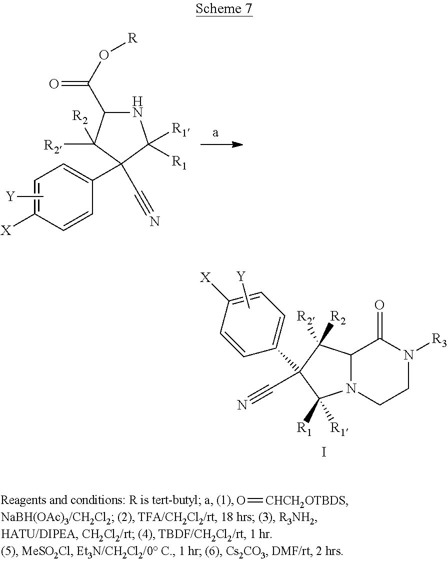

Converting a Compound of Formula I that Bears a Basic Nitrogen into a Pharmaceutically Acceptable Acid Addition Salt

The optional conversion of a compound of formula I that bears basic nitrogen into a pharmaceutically acceptable acid addition salt can be effected by conventional means. For example, the compound can be treated with an inorganic acid such as for example hydrochloric acid, hydrobromic acid, sulfuric acid, nitric acid, phosphoric acid, or with an appropriate organic acid such as acetic acid, citric acid, tartaric acid, methanesulfonic acid, p-toluene sulfonic acid, or the like.

Converting a Compound of Formula I that Bears a Carboxylic Acid Group into a Pharmaceutically Acceptable Alkali Metal Salt

The optional conversion of a compound of formula I that bears a carboxylic acid group into a pharmaceutically acceptable metal salt can be effected by conventional means. For example, the compound can be treated with an inorganic base such as lithium hydroxide, sodium hydroxide, potassium hydroxide, or the like.

### Crystal Forms

When the compounds of the invention are solids, it is understood by those skilled in the art that these compounds, and their salts, may exist in different crystal or polymorphic forms, all of which are intended to be within the scope of the present invention and specified formulas.

### EXAMPLES

The compounds of the present invention may be synthesized according to known techniques. The following examples and references are provided to aid the understanding of the present invention. The examples are not intended, however, to limit the invention, the true scope of which is set forth in the appended claims. The names of the final products in the examples were generated using Isis AutoNom 2000.

### Abbreviations Used in the Examples

### HRMS: High Resolution Mass Spectrometry

### LCMS: Liquid Chromatography Mass Spectrometry

HATU: 2-(7-Azabenzotriazol-1-yl)-n,n,n′,n′-tetramethyluronium hexafluorophosphate
RT (or rt) Room temperature
DBU: 1,8-Diazabicyclo[5,4,0]undec-7-ene
DIBAL: Diisobutylalumiunum hydride
iPA: Isopropyl alcohol
ASDI: ASDI-Intermediates (company name)
RP-HPLC: Reverse phase HPLC

### Min: Minutes

### H or hrs: Hours

### GST; Glutathione S-transferase

TRF: Time resolved fluorescence

### Example 1

### Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-methoxy-benzoic acid

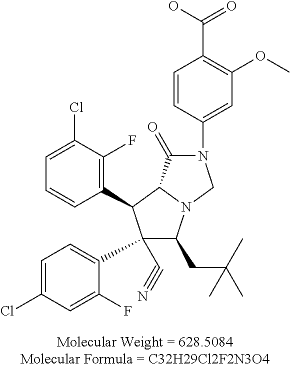

4-((2R,3S,4R,5S)-3-(3-chloro-2-fluorophenyl)-4-(4-chloro-2-fluorophenyl)-4-cyano-5-neopentylpyrrolidine-2-carboxamido)-2-methoxybenzoic acid (WO 2010031713 A1, 50 mg, 0.081 mmol) was dissolved in 10 mL of THF. Formaldehyde (Aldrich, 37% in water, 0.25 mL) was added and the mixture was stirred at 75° C. overnight. The reaction mixture was concentrated to remove some THF. Water was added to form a suspension. The solid was collected by filtration, washed with water and diethyl ether (5 mL) and dried under high vacuum. It was freeze dried to give the desired product. (48 mg, 94%). LCMS (ES⁺) m/z Calcd, for C₃₂H₂₉Cl₂F₂N₃O₄ [(M+H)⁺]: 628. Found: 628.

### Example 2

### Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-benzoic acid

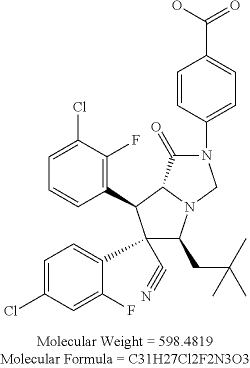

In a 20 mL sealed tube, 4-((2R,3S,4R,5S)-3-(3-chloro-2-fluorophenyl)-4-(4-chloro-2-fluorophenyl)-4-cyano-5-neopentylpyrrolidine-2-carboxamido)benzoic acid (WO 2010031713 A1, 200 mg, 0.341 mmol) was combined with DME (6 mL) to give a colorless solution. Formaldehyde (554 mg, 6.82 mmol) in water was added and the reaction was stirred at 85° C. for 20 hr.

The reaction mixture was concentrated to remove some DME, water was added to form a suspension. The solid was collected by filtration, washed with water and diethyl ether (5 mL) and dried with high vacuum. It was freeze dried to give a white solid as desired product (180 mg, 87%). LCMS (ES⁺) m/z Calcd for C₃₁H₂₇Cl₂F₂N₃O₃ [(M+H)⁺]: 598. Found: 598.

### Example 3

### Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-methoxy-benzoic acid

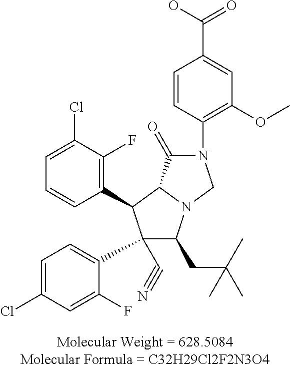

4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoic acid (WO 2010031713 A1, 200 mg, 0.324 mmol) was dissolved in 10 mL of THF. Formaldehyde (Aldrich, 37% in water, 300 mg, 3.7 mmol) was added and the mixture was stirred at 85° C. overnight. The reaction mixture was concentrated to remove some THF. Water was added to form a suspension. The solid was collected by filtration, washed with water and diethyl ether (5 mL) and dried under high vacuum. The solid was dissolved in DMSO and was purified by HPLC (50%-100% ACN/water/TFA). The fractions were combined, concentrated and freeze dried to give a white foam as desired product (118 mg, 58%). LCMS (ES⁺) m/z Calcd for C₃₂H₂₉Cl₂F₂N₃O₄ [(M+H)⁺]: 628. Found: 628.

### Example 4

### Preparation of [(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-acetic acid

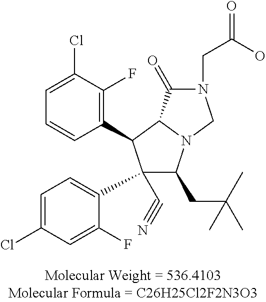

In a 20 mL pressure tube, {[(2R,3S,4R,5S)-4-(4-Chloro-2-fluoro-phenyl)-3-(3-chloro-2-fluoro-phenyl)-4-cyano-5-(2,2-dimethyl-propyl)-pyrrolidine-2-carbonyl]-amino}-acetic acid (WO 2010031713 A1, 30 mg, 0.0572 mmol) was combined with DME (2 mL) to give a colorless solution. Formaldehyde in water (0.5 mL) was added and the reaction was stirred at 90° C. for 2 hr.

The reaction mixture was concentrated to remove some DME, added water (8 mL), and extracted with EtOAc (3×10 mL). The organic layer was dried over Na₂SO₄ and concentrated in vacuo. The crude material was purified by preparative HPLC (30-100% ACN/water/0.1% TFA). The fractions were combined and freeze dried to give a white foam as desired product (11.2 mg, 36%). LCMS (ES⁺) m/z Calcd for C₂₆H₂₅Cl₂F₂N₃O₃ [(M+H)⁺]: 536. Found: 536.

### Example 5

Preparation of 2-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-N,N-bis-(2-methoxy-ethyl)-acetamide

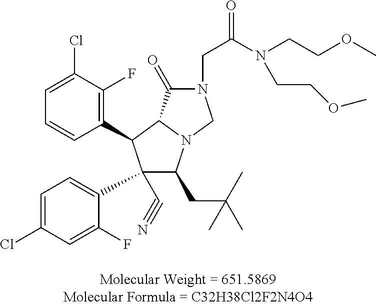

In a 20 mL pressure tube, (2R,3S,4R,5S)—N-(2-(bis(2-methoxyethyl)amino)-2-oxoethyl)-3-(3-chloro-2-fluorophenyl)-4-(4-chloro-2-fluorophenyl)-4-cyano-5-neopentylpyrrolidine-2-carboxamide (WO 2010031713 A1, 30 mg, 46.9 μmol) was combined with DME (2.00 mL) to give a colorless solution. Formaldehyde in water (0.5 mL) was added and the reaction was stirred at 90° C. for 2 hr.

The reaction mixture was concentrated to remove some DME, added water (8 mL), and extracted with EtOAc (3×10 mL). The organic layers were dried over Na₂SO₄ and concentrated in vacuum. The crude material was purified by preparative HPLC (30-100% ACN/water/0.1% TFA). The fractions were combined and freeze dried to give a white foam as desired product (9.6 mg, 31%). LRMS (ES⁺) m/z Calcd for C₃₂H₃₈Cl₂F₂N₄O₄ [(M+H)⁺]: 651. Found: 651.

### Example 6

### Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-hydroxy-benzoic acid

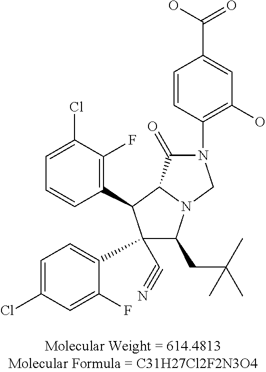

In a 20 mL scintillation vial, 4-((2R,3S,4R,5S)-3-(3-chloro-2-fluorophenyl)-4-(4-chloro-2-fluorophenyl)-4-cyano-5-neopentylpyrrolidine-2-carboxamido)-3-hydroxybenzoic acid (WO 2010031713 A1, 30 mg, 49.8 μmol) was combined with DME (2 mL) to give a colorless solution. Formaldehyde in water (0.5 ml) was added and the reaction was stirred at 60° C. for 5 hrs.

The reaction mixture was concentrated to remove some DME, added water (8 mL), and extracted with EtOAc (3×10 mL). The organic layers were dried over Na₂SO₄ and concentrated in vacuum. The residue was recrystallized from CH₂Cl₂, filtered and washed with CH₂Cl₂/Hexanes to give a white solid as desired product (25.2 mg, 74%). LCMS (ES⁺) m/z Calcd for C₃₁H₂₇Cl₂F₂N₃O₄ [(M+H)⁺]: 614. Found: 614.

### Example 7

### Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-trifluoromethyl-benzoic acid

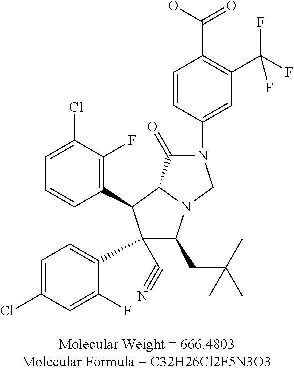

In a 20 mL scintillation vial, 4-((2R,3S,4R,5S)-3-(3-chloro-2-fluorophenyl)-4-(4-chloro-2-fluorophenyl)-4-cyano-5-neopentylpyrrolidine-2-carboxamido)-2-(trifluoromethyl)benzoic acid (WO 2010031713 A1, 30 mg, 45.8 μmol) was combined with DME (2.00 mL) to give a colorless solution. Formaldehyde in water (0.5 mL) was added and the reaction was stirred at 60° C. for 5 hr.

The reaction mixture was concentrated to remove some DME, added water (8 mL), and extracted with EtOAc (3×10 mL). The organic layers were dried over Na₂SO₄ and concentrated in vacuum. The crude material was purified by flash chromatography (silica gel, 12 g, 5% to 7% MeOH in DCM) to give a white solid, which was purified again by HPLC (50-100% ACN/water/0.1% TFA) to give a white foam after freeze drying as desired product (24.5 mg, 79%). LCMS (ES⁺) m/z Calcd for C₃₂H₂₆Cl₂F₅N₃O₃ [(M+H)⁺]: 666. Found: 666.

### Example 8

### Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-fluoro-benzoic acid

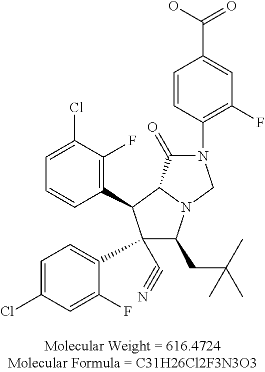

In a 20 mL sealed tube, 4-((2R,3S,4R,5S)-3-(3-chloro-2-fluorophenyl)-4-(4-chloro-2-fluorophenyl)-4-cyano-5-neopentylpyrrolidine-2-carboxamido)-3-fluorobenzoic acid (WO 2010031713 A1, 38 mg, 62.9 μmol) was combined with DME (2 mL) to give a colorless solution. Formaldehyde in water (0.5 mL) was added and the reaction was stirred at 60° C. for 5 hr.

The reaction mixture was concentrated to remove some DME, added water (8 mL), and extracted with EtOAc (3×10 mL). The organic layers were dried over Na₂SO₄ and concentrated in vacuum. The crude material was purified by flash chromatography (silica gel, 12 g, 5% to 7% MeOH in DCM) to give a white solid. which was purified again by HPLC (50-100% ACN/water/0.1% TFA) to give a powder after freeze drying as desired product (26.7 mg, 68%). LCMS (ES⁺) m/z Calcd for C₃₁H₂₆Cl₂F₃N₃O₃ [(M+H)⁺]: 616. Found: 616.

### Example 9

### Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-trifluoromethoxy-benzoic acid

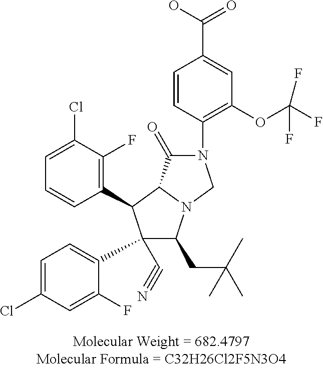

In a 20 mL scintillation vial, 4-((2R,3S,4R,5S)-3-(3-chloro-2-fluorophenyl)-4-(4-chloro-2-fluorophenyl)-4-cyano-5-neopentylpyrrolidine-2-carboxamido)-3-(trifluoromethoxy)benzoic acid (WO 2010031713 A1, 30 mg, 44.7 μmol) was combined with DME (2 mL) to give a colorless solution. Formaldehyde in water (0.5 mL) was added and the reaction was stirred at 60° C. for 5 hr.

The reaction mixture was concentrated to remove some DME, added water (8 mL), and extracted with EtOAc (3×10 mL). The organic layers were dried over Na₂SO₄ and concentrated in vacuum. The crude material was purified by flash chromatography (silica gel, 12 g, 5% to 7% MeOH in DCM) to give a white solid which was purified again by HPLC (50-100% ACN/water/0.1% TFA) to give a powder after freeze drying as desired product (20.6 mg, 67%). LCMS (ES⁺) m/z Calcd for C₃₂H₂₆Cl₂F₅N₃O₄ 682. Found: 682.

### Example 10

### Preparation of (5S,6R,7S,7aR)-7-(3-chloro-2-fluoro-phenyl)-6-(4-chloro-2-fluoro-phenyl)-5-(2,2-dimethyl-propyl)-2-(3-hydroxy-phenyl)-1-oxo-hexahydro-pyrrolo[1,2-c]imidazole-6-carbonitrile

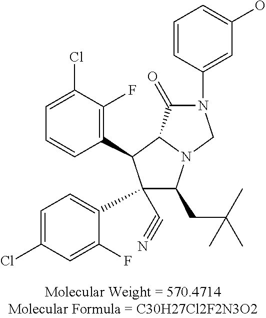

In a 20 mL scintillation vial, (2R,3S,4R,5S)-3-(3-chloro-2-fluorophenyl)-4-(4-chloro-2-fluorophenyl)-4-cyano-N-(3-hydroxyphenyl)-5-neopentylpyrrolidine-2-carboxamide (WO 2010031713 A1, 50 mg, 62.7 μmol) was combined with DME (2.00 mL) to give a colorless solution. Formaldehyde in water (0.5 mL) was added, followed by 1M HCl (96.0 mg, 80 μL, 80.0 μmol) and the reaction was stirred at 60° C. for 5 hr.

The reaction mixture was concentrated to remove some DME, added water (8 mL), and extracted with EtOAc (3×10 mL). The organic layers were dried over Na₂SO₄ and concentrated in vacuum. The crude material was purified by HPLC (50-100% ACN/water/0.1% TFA) to give a white foam after freeze drying as desired product (18.3 mg, 51%). LCMS (ES⁺) m/z Calcd for C₃₀H₂₇Cl₂F₂N₃O₂ [(M+H)]: 570. Found: 570.

### Example 11

### Preparation of (5S,6R,7S,7aR)-7-(3-chloro-2-fluoro-phenyl)-6-(4-chloro-2-fluoro-phenyl)-5-(2,2-dimethyl-propyl)-2-(2-hydroxy-phenyl)-1-oxo-hexahydro-pyrrolo[1,2-c]imidazole-6-carbonitrile

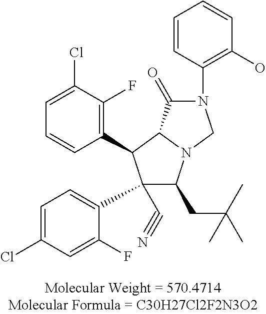

In a 20 mL scintillation vial, (2R,3S,4R,5S)-3-(3-chloro-2-fluorophenyl)-4-(4-chloro-2-fluorophenyl)-4-cyano-N-(2-hydroxyphenyl)-5-neopentylpyrrolidine-2-carboxamide (WO 2010031713 A1, 30 mg, 53.7 μmol) was combined with DME (2.00 mL) to give a colorless solution. Formaldehyde in water (0.5 mL) was added, followed by 1M HCl (80 μL, 80.0 μmol) and the reaction was stirred at 60° C. for 5 hrs.

The reaction mixture was concentrated to remove some DME, added water (8 mL), and extracted with EtOAc (3×10 mL). The organic layers were dried over Na₂SO₄ and concentrated in vacuo. The crude material was purified by preparative HPLC (50-100% ACN/Water/0.1% TFA) to give a white foam after freeze drying as desired product (24.6 mg, 79%). LCMS (ES⁺) m/z Calcd for C₃₀H₂₇Cl₂F₂N₃O₂ [(M+H)⁺]: 570. Found: 570.

### Example 12

### Preparation of (5S,6R,7S,7aR)-7-(3-chloro-2-fluoro-phenyl)-6-(4-chloro-2-fluoro-phenyl)-5-(2,2-dimethyl-propyl)-2-(2-fluoro-4-methoxy-phenyl)-1-oxo-hexahydro-pyrrolo[1,2-c]imidazole-6-carbonitrile

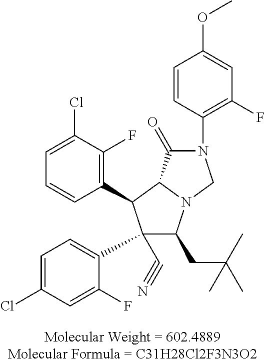

In a 20 mL scintillation vial, (2R,3S,4R,5S)-3-(3-chloro-2-fluorophenyl)-4-(4-chloro-2-fluorophenyl)-4-cyano-N-(2-fluoro-4-methoxyphenyl)-5-neopentylpyrrolidine-2-carboxamide (WO 2010031713 A1, 50 mg, 84.7 μmol) was combined with DME (2.00 ml) to give a colorless solution. Formaldehyde in water (0.5 mL) was added, followed by 1M HCl (100 μA, 100 μmol). The reaction mixture was heated to 60° C. and stirred for 3 days.

The reaction mixture was poured into 10 mL H₂O and extracted with EtOAc (3×15 mL). The organic layers were combined, washed with sat NaCl (1×10 mL), dried over Na₂SO₄ and concentrated in vacuum. The crude material was purified by flash chromatography (silica gel, 12 g, 3% to 6% EtOAc in DCM) to give a white foam after freeze drying as desired product (43.5 mg, 85%). LCMS (ES⁺) m/z Calcd for C₃₁H₂₈Cl₂F₃N₃O₂ [(M+H)⁺]: 602. Found: 602.

### Example 13

Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzoic acid methyl ester

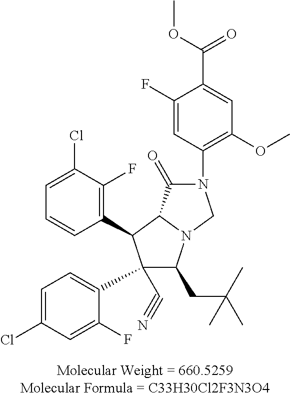

In a 20 mL scintillation vial, methyl 4-((2R,3S,4R,5S)-3-(3-chloro-2-fluorophenyl)-4-(4-chloro-2-fluorophenyl)-4-cyano-5-neopentylpyrrolidine-2-carboxamido)-2-fluoro-5-methoxybenzoate (WO 2010031713 A1, 20 mg, 30.8 μmol) was combined with DME (2.00 mL) to give a colorless solution. Formaldehyde in water (0.3 mL) was added, followed by 1M HCl (20 μL, 20.0 μmol). The reaction mixture was heated to 60° C. and stirred overnight.

The reaction mixture was concentrated and the crude material was purified by preparative HPLC (50-100% ACN/water/0.1% TFA) to give a white foam after freeze drying as desired product (13.2 mg, 65%). LCMS (ES⁺) m/z Calcd for C₃₃H₃₀Cl₂F₃N₃O₄ [(M+H)⁺)]: 660. Found: 660.

### Example 14

### Preparation of 4-[(5S,6R,7S,7aR)-7-(3-chloro-2-fluoro-phenyl)-6-(4-chloro-2 fluoro-phenyl)-6-cyano-5-(2,2-dimethyl-propyl)-1-oxo-tetrahydro-pyrrolo[1,2-c]imidazol-2-yl]-3-methoxy-benzamide

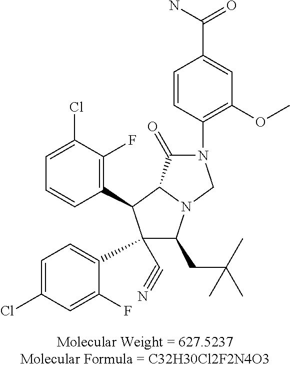

In a 10 mL round-bottomed flask, 4-((5S,6R,7S,7aR)-7-(3-chloro-2-fluorophenyl)-6-(4-chloro-2-fluorophenyl)-6-cyano-5-neopentyl-1-oxo-1H-pyrrolo[1,2-c]imidazol-2(3H,5H,6H,7H,7aH)-yl)-3-methoxybenzoic acid (WO 2010031713 A1, 40 mg, 63.6 μmol) was combined with CH₂Cl₂ (3 mL) to give a suspension. DIPEA (16.5 mg, 22.2 μL, 127 μmol) and HATU (26.6 mg, 70.0 μmoL) were added. The reaction was stirred for 2 minutes and 2M ammonia (159 μL, 318 μmol) in methanol was added. The reaction mixture was stirred at rt for 2 hrs.

The crude material was purified by preparative HPLC (45-85% ACN/water/0.1% TFA). The fractions were combined to give a white foam after freeze drying as desired product (30.7 mg, 77%). LCMS (ES⁺) m/z Calcd for C₃₂H₃₀Cl₂F₂N₄O₃ [(M+H)⁺]: 627. Found: 627.

### Example 15

Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-methoxy-benzoic acid methyl ester

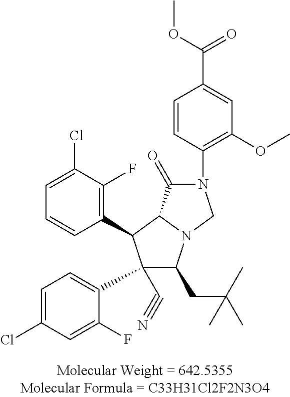

In a 20 mL scintillation vial, methyl 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (WO 2010031713 A1, 150 mg, 238 μmol) was combined with DME (5.00 mL) to give a colorless solution. Formaldehyde in water (1 mL) was added, followed by 1M HCl (200 μL, 200 μmol). The reaction mixture was heated to 60° C. and stirred overnight.

The reaction mixture was poured into 15 mL of H₂O and extracted with EtOAc (3×25 mL). The organic layers were dried over Na₂SO₄ and concentrated in vacuum.

The crude material was purified by preparative HPLC (50-100% ACN/water/0.1 TFA) to give a white foam after freeze drying as desired product (91 mg, 59%). LCMS (ES⁺) m/z Calcd for C₃₃H₃₁Cl₂F₂N₃O₄ [(M+H)⁺]: 642. Found: 642.

### Example 16

### Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzamide

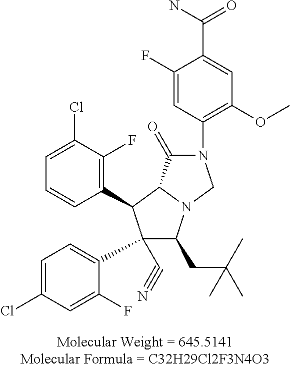

In a 15 mL round-bottomed flask, 4-((5S,6R,7S,7aR)-7-(3-chloro-2-fluorophenyl)-6-(4-chloro-2-fluorophenyl)-6-cyano-5-neopentyl-1-oxo-1H-pyrrolo[1,2-c]imidazol-2(3H,5H,6H,7H,7aH)-yl)-2-fluoro-5-methoxybenzoic acid (Example 18, 55 mg, 85.1 μmol) was combined with CH₂Cl₂ (3 mL) to give a suspension. DIPEA (22.0 mg, 29.7 μL, 170 μmol) and HATU (35.6 mg, 93.6 μmol) were added. The reaction was stirred for 2 minutes and 2M ammonia (213 μL, 425 μmol) in methanol was added. The reaction mixture was stirred at rt overnight.

The reaction mixture was concentrated and the crude material was purified by preparative HPLC (45-100% ACN/water/0.1% TFA). The fractions were combined to give a white foam after freeze drying as desired product (49 mg, 88%). LCMS (ES⁺) m/z Calcd for C₃₂H₂₉Cl₂F₃N₄O₃ [(M+H)⁺]: 645. Found: 645.

### Example 17

### Preparation of 5-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-pyridine-2-carboxylic acid

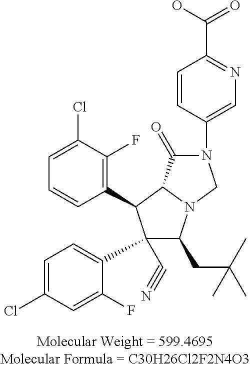

In a 20 mL scintillation vial, 5-((2R,3S,4R,5S)-3-(3-chloro-2-fluorophenyl)-4-(4-chloro-2-fluorophenyl)-4-cyano-5-neopentylpyrrolidine-2-carboxamido)picolinic acid (WO 2010031713 A1, 30 mg, 51.1 μmol) was combined with DME (5.00 mL) to give a colorless solution. Formaldehyde in water (0.3 mL) was added, followed by 1M HCl (50 μL, 50.0 μmol). The reaction mixture was heated to 60° C. and stirred overnight.

The reaction mixture was concentrated and the crude material was purified by preparative HPLC (50-100% ACN/water/0.1% TFA to give a white foam after freeze drying as desired product (16.1 mg, 52%). LCMS (ES⁺) m/z Calcd for C₃₀H₂₆Cl₂F₂N₄O₃ [(M+H)⁺]: 599. Found: 599.

### Example 18

### Preparation of 4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzoic acid

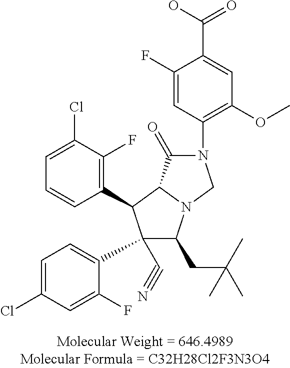

In a 20 mL scintillation vial, 4-((2R,3S,4R,5S)-3-(3-chloro-2-fluorophenyl)-4-(4-chloro-2-fluorophenyl)-4-cyano-5-neopentylpyrrolidine-2-carboxamido)-2-fluoro-5-methoxybenzoic acid (WO 2010031713 A1, 230 mg, 181 μmol) was combined with DME (4 mL) to give a light brown solution. Formaldehyde in water (0.5 mL) was added, followed by 1M HCl (50 μL, 50.0 μmol). The reaction mixture was heated to 60° C. and stirred overnight.

The reaction mixture was concentrated and the crude material was purified by preparative HPLC (50-100% ACN/water/0.1% TFA) to give a white solid as desired product (88.3 mg, 74%). LCMS (ES⁺) m/z Calcd for C₃₂H₂₈Cl₂F₃N₃O₄ [(M+H)⁺]: 646. Found: 646.

### Example 19

Preparation of (5S,6R,7S,7aR)-7-(3-chloro-2-fluoro-phenyl)-6-(4-chloro-2-fluoro-phenyl)-5-(2,2-dimethyl-propyl)-2-(1-methanesulfonyl-piperidin-4-yl)-1-oxo-hexahydro-pyrrolo[1,2-c]imidazole-6-carbonitrile

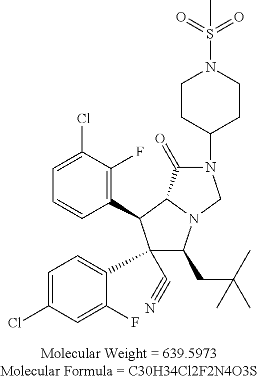

In a 20 mL scintillation vial, (2R,3S,4R,5S)-3-(3-chloro-2-fluorophenyl)-4-(4-chloro-2-fluorophenyl)-4-cyano-N-(1-(methylsulfonyl)piperidin-4-yl)-5-neopentylpyrrolidine-2-carboxamide (WO 2010031713 A1, 30 mg, 47.8 μmol) was combined with DME (2.00 mL) to give a colorless solution. Formaldehyde in water (0.5 mL) was added and the reaction was stirred at 60° C. for 5 hr. The reaction mixture was concentrated and the crude was purified by HPLC (50-100% ACN/water/0.1% TFA) to give a white solid (7.1 mg, 23%). LCMS (ES⁺) m/z Calcd for C₃₀H₃₄Cl₂F₂N₄O₃S [(M+H)⁺]: 639. Found: 639.

### Example 20

### Preparation of (5S,6R,7S,7aR)-2-(1-acetyl-piperidin-4-yl)-7-(3-chloro-2-fluoro-phenyl)-6-(4-chloro-2-fluoro-phenyl)-5-(2,2-dimethyl-propyl)-1-oxo-hexahydro-pyrrolo[1,2-c]imidazole-6-carbonitrile

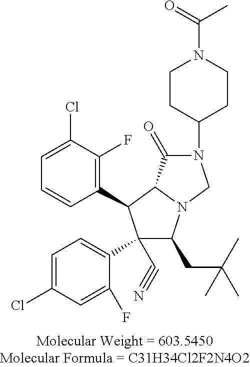

In a 20 mL scintillation vial, (2R,3S,4R,5S)-N-(1-acetylpiperidin-4-yl)-3-(3-chloro-2-fluorophenyl)-4-(4-chloro-2-fluorophenyl)-4-cyano-5-neopentylpyrrolidine-2-carboxamide (WO 2010031713 A1, 30 mg, 50.7 μmol) was combined with DME (2.00 mL) to give a colorless solution. Formaldehyde in water (0.5 mL) was added and the reaction was stirred at 60° C. for 5 hr.

The reaction mixture was concentrated and the crude was purified by HPLC (50-100% ACN/water/0.1% TFA) to give a white solid (7.7 mg, 25%). LCMS (ES⁺) m/z Calcd for C₃₁H₃₄Cl₂F₂N₄O₂ [(M+H)⁺]: 603. Found: 603.

### Example 21

### Preparation of rac-(5S,6R,7R,7aR)-7-(3-chloro-phenyl)-6-(4-chlorophenyl)-5-(2,2-dimethyl-propyl)-2-methyl-1,3-dioxo-hexahydro-pyrrolo[1,2-c]imidazole-6-carbonitrile

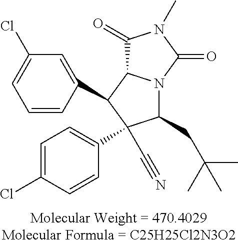

A solution of rac-(2R,3R,4R,5S)-3-(3-chloro-phenyl)-4-(4-chloro-phenyl)-4-cyano-5-(2,2-dimethyl-propyl)-pyrrolidine-2-carboxylic acid tert-butyl ester (WO 2010031713 A1, 97.5 mg, 0.20 mmol) and MeNCO (Aldrich, 114.2 mg, 2.0 mmol) in THF (1.0 mL) was heated at 150° C. for 10 min with CEM microwave reactor. To the mixture was then added a few drops of 2 N H₂SO₄ and the mixture was further heated at 150° C. for 10 min with CEM microwave reactor. The reaction mixture was extracted with AcOEt. The organic phase was separated, filtered and dried over Na₂SO₄. The mixture was then concentrated and purified by flash column to give a white amorphous (19.5 mg, 20.7%).

HRMS (ES⁺) m/z Calcd for C₂₅H₂₅Cl₂N₃O₂ [(M+H)⁺]: 470.1397. Found: 470.1394.

### Example 22

### Preparation of rac-(5S,6R,7R,7aR)-7-(3-chloro-phenyl)-6-(4-chlorophenyl)-5-(2,2-dimethyl-propyl)-2-ethyl-1,3-dioxo-hexahydro-pyrrolo[1,2-c]imidazole-6-carbonitrile

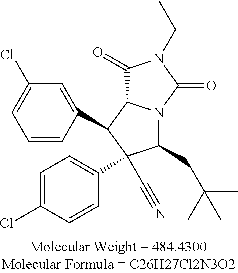

In a manner similar to Example 21 rac-(2R,3R,4R,5S)-3-(3-chloro-phenyl)-4-(4-chloro-phenyl)-4-cyano-5-(2,2-dimethyl-propyl)-pyrrolidine-2-carboxylic acid tert-butyl ester (WO 2010031713 A1, 97.5 mg, 0.20 mmol) and EtNCO (Aldrich, 142.2 mg, 2.0 mmol) in THF (1.0 mL) was heated at 180° C. for 10 min followed by treatment with 2 NH₂SO₄ at 180° C. for 5 min with CEM microwave reactor to give a white amorphous (45.8 mg, 43.7%). HRMS (ES⁺) m/z Calcd for C₂₆H₂₇Cl₂N₃O₂ [(M+H)⁺]: 484.1553. Found: 484.1553.

### Example 23

### Preparation of rac-(5R,6R,7R,7aR)-7-(3-chloro-phenyl)-6-(4-chlorophenyl)-5-(2,2-dimethyl-propyl)-2-ethyl-1,3-dioxo-hexahydro-pyrrolo[1,2-c]imidazole-6-carbonitrile

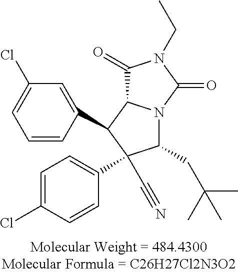

In a manner similar to Example 21 rac-(2R,3R,4R,5R)-3-(3-chloro-phenyl)-4-(4-chloro-phenyl)-4-cyano-5-(2,2-dimethyl-propyl)-pyrrolidine-2-carboxylic acid tert-butyl ester (WO 2010031713 A1, 97.5 mg, 0.20 mmol) and EtNCO (Aldrich, 142.2 mg, 2.0 mmol) in THF (1.0 mL) was heated at 180° C. for 10 min followed by treatment with 2 NH₂SO₄ at 180° C. for 5 min with CEM microwave reactor to give a white amorphous (48.8 mg, 50.4%). HRMS (ES⁺) m/z Calcd for C₂₆H₂₇Cl₂N₃O₂ [(M+H)⁺]: 484.1553. Found: 484.1553.

### Example 24

### Preparation of rac-3-[(5S,6R,7R,7aR)-7-(3-chloro-phenyl)-6-(4-chlorophenyl)-6-cyano-5-(2,2-dimethyl-propyl)-1,3-dioxo-tetrahydro-pyrrolo[1,2-c]imidazole-2-yl]-propionic acid ethyl ester

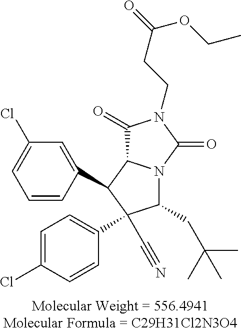

In a manner similar to Example 21 rac-(2R,3R,4R,5S)-3-(3-chloro-phenyl)-4-(4-chloro-phenyl)-4-cyano-5-(2,2-dimethyl-propyl)-pyrrolidine-2-carboxylic acid tert-butyl ester (WO 2010031713 A1, 97.5 mg, 0.20 mmol) and 3-isocyanato-propionic acid ethyl ester (Aldrich, 143.2 mg, 1.0 mmol) in THF (1.0 mL) was heated at 150° C. for 30 min. followed by treatment with 2 NH₂SO₄ at 150° C. for 60 min. with CEM microwave reactor to give a white amorphous (67.8 mg, 60.9%). HRMS (ES⁺) m/z Calcd for C₂₉H₃₁Cl₂N₃O₄ [(M+H)⁺]: 556.1765. Found: 556.1762.

### Example 25b

### Preparation of rac-(2R,3R,4R,5S)-3-(3-chloro-phenyl)-4-(4-chloro-phenyl)-4-cyano-5-(2,2-dimethyl-propyl)-1-[(4-hydroxy-butylcarbamoyl)-methyl]-pyrrolidine-2-carboxylic acid tert-butyl ester

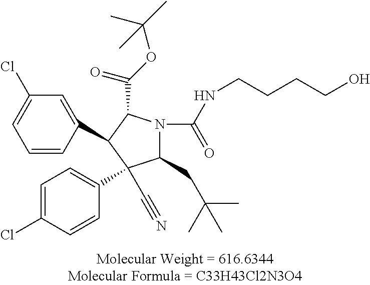

To a mixture of rac-(2R,3R,4R,5S)-3-(3-chloro-phenyl)-4-(4-chloro-phenyl)-4-cyano-5-(2,2-dimethyl-propyl)-pyrrolidine-2-carboxylic acid tert-butyl ester (WO 2010031713 A1, 48.8 mg, 0.10 mmol) and TEA (0.2 mL, excess) in CH₂Cl₂ (2.0 mL) was added phosgene (Aldrich, 1.0 M in toluene, 0.15 mL) and the reaction mixture was stirred at rt for 30 min. 3-amino-1-propanol was then added by injection. The mixture was stirred at rt for another 30 min then diluted with CH₂Cl₂ and washed with water, brine. The organic phase was separated, filtered and dried over Na₂SO₄. The mixture was then concentrated and purified by flash column (5-50% AcOEt in Hex) to give a white amorphous (36.5 mg, 62%). HRMS (ES⁺) m/z Calcd for C₃₁H₃₉Cl₂N₃O₄ [(M+H)⁺]: 588.2391. Found: 588.2392.

### Example 25b

### Preparation of rac-(5S,6R,7R,7aR)-7-(3-chloro-phenyl)-6-(4-chloro-phenyl)-5-(2,2-dimethyl-propyl)-2-(4-hydroxy-butyl)-1,3-dioxo-hexahydro-pyrrolo[1,2-c]imidazole-6-carbonitrile

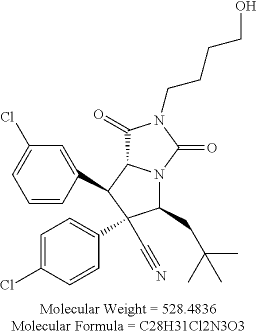

To a mixture of rac-(2R,3R,4R,5S)-3-(3-chloro-phenyl)-4-(4-chloro-phenyl)-4-cyano-5-(2,2-dimethyl-propyl)-1-[(4-hydroxy-butylcarbamoyl)-methyl]-pyrrolidine-2-carboxylic acid tert-butyl ester (WO 2010031713 A1, 29.5 mg, 0.05 mmol) in CH₂Cl₂ (1.0 mL) was added a few drops of 2 NH₂SO₄ and the mixture was further heated at 150° C. for 10 min with CEM microwave reactor. The reaction mixture was extracted with AcOEt. The organic phase was separated, filtered and dried over Na₂SO₄. The mixture was then concentrated and purified by flash column to give a white amorphous (10.5 mg, 40.6%)

HRMS (ES⁺) m/z Calcd for C₂₇H₂₉Cl₂N₃O₃ [(M+H)⁺]: 514.1659. Found: 514.1660.

### Example 26

### Preparation of rac-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid methyl ester

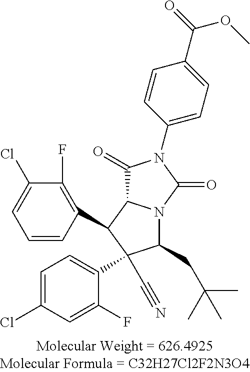

In a 10 mL pressure tube, rac (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (WO 2010031713 A1, 93.4 mg, 0.20 mmol) were combined with methyl 4-isocyanatobenzoate (Aldrich, 72.3 mg, 0.40 mmol) in CH₂Cl₂ (5.0 mL) to give a white suspension. The reaction mixture was heated at 120° C. for 10 min with CEM microwave reactor. The crude reaction mixture was concentrated in vacuo and the residue was purified by flash chromatography (silica gel, 12 g, 1% to 25% EtOAc in hexanes) to give a white powder (115.6 mg, 92.3%).

HRMS (ES⁺) m/z Calcd for C₃₂H₂₇Cl₂F₂N₃O₄ [(M+H)⁺]: 626.1420. Found: 626.1418.

### Example 27

### Preparation of rac-4-[(5S,6R,7S,7aR)-7-(3-chloro-2-fluoro-phenyl)-6-(4-chloro-2-fluoro-phenyl)-2-(4-cyano-phenyl)-5-(2,2-dimethyl-propyl)-1,3-dioxo-hexahydro-pyrrolo[1,2-c]imidazole-6-carbonitrile

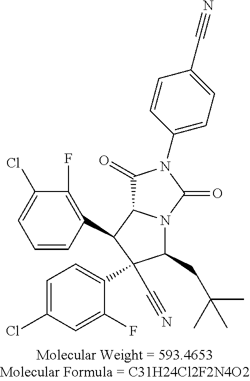

In a 10 mL pressure tube, rac (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (WO 2010031713 A1, 46.7 mg, 0.10 mmol) were combined with 4-isocyanatobenzonitrile (Aldrich, 28.8 mg, 0.20 mmol) in CH₂Cl₂ (5.0 mL) to give a white suspension. The reaction mixture was stirred at rt overnight. The crude reaction mixture was concentrated in vacuo and the residue was purified by flash chromatography (silica gel, 12 g, 1% to 25% EtOAc in hexanes) to give a white powder (53.5 mg, 90.2%).

HRMS (ES⁺) m/z Calcd for C₃₁H₂₄Cl₂F₂N₄O₂ [(M+H)⁺]: 593.1317. Found: 593.1313.

### Example 28

### Preparation of rac-4-[(5S,6R,7S,7aR)-7-(3-chloro-2-fluoro-phenyl)-6-(4-chloro-2-fluoro-phenyl)-5-(2,2-dimethyl-propyl)-1,3-dioxo-2-phenyl-hexahydro-pyrrolo[1,2-c]imidazole-6-carbonitrile

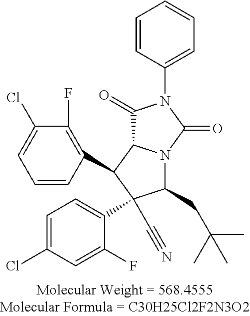

In a 10 mL pressure tube, rac (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (WO 2010031713 A1, 46.7 mg, 0.10 mmol) were combined with 4-isocyanatobenzene (Aldrich, 23.8 mg, 0.20 mmol) in CH₂Cl₂ (5.0 mL) to give a white suspension. The reaction mixture was stirred at rt overnight. The crude reaction mixture was concentrated in vacuo and the residue was purified by flash chromatography (silica gel, 12 g, 1% to 25% EtOAc in hexanes) to give a white powder (13.6 mg, 23.9%).

HRMS (ES⁺) m/z Calcd for C₃₀H₂₅Cl₂F₂N₃O₂ [(M+H)⁺]: 568.1365. Found: 536.1364.

### Example 29

### Preparation of rac-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid

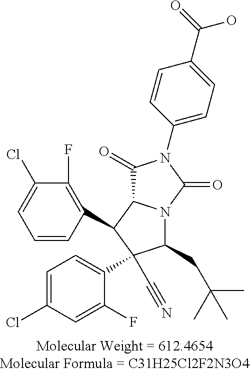

In a 10 mL pressure tube was placed rac-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid methyl ester (125.0 mg, 0.20 mmol) in MeCN, and AlI₃ (Aldrich, 815.0 mg, 2.0 mmol) was added in portions. The reaction mixture was heated to 160° C. overnight. The crude reaction mixture was poured into 25 mL ice-water and extracted with EtOAc. The organic layers were combined, washed with Na₂SO₃ (1×20 mL), H₂O (3×15 mL), and sat NaCl (1×15 mL). The crude reaction mixture was concentrated in vacuo. The crude material was purified by flash chromatography (silica gel, 12 g, 5% to 80% EtOAc in hexanes) to give a white powder (155.6 mg, 45.4%). HRMS (ES⁺) m/z Calcd for C₃₁H₂₅Cl₂F₂N₃O₄ [(M+H)⁺]: 612.1263. Found: 612.1264.

### Example 30

### Preparation of chiral-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid

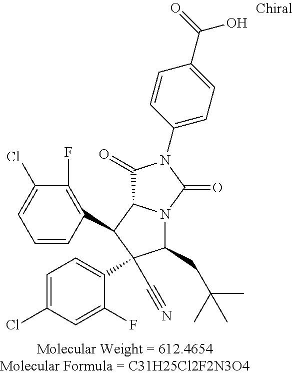

In a manner similar to Example 26 and 29 chiral-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (WO 2010031713 A1, 93.4 mg, 0.20 mmol) were reacted with methyl 4-isocyanatobenzoate (Aldrich, 72.3 mg, 0.40 mmol) to give chiral-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid methyl ester (82.5 mg, 65.9%). The methyl ester (70 mg, 0.11 mmol) was treated with AlI₃ (Aldrich, 456 mg, 1.1 mmol) at 180° C. for 30 minutes with CEM microwave reactor. The crude reaction mixture was poured into 25 mL ice-water and extracted with EtOAc. The organic layers were combined, washed with Na₂SO₃ (1×20 mL), H₂O (3×15 mL), and sat NaCl (1×15 mL). The crude reaction mixture was concentrated in vacuo to give a white powder (65.1 mg, 95.1%). HRMS (ES⁺) m/z Calcd for C₃₁H₂₅Cl₂F₂N₃O₄-[(M+H)⁺]: 612.1263. Found: 612.1264.

### Example 31

### Preparation of rac-3-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid ethyl ester

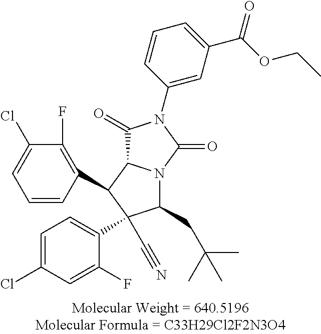

In a 10 mL pressure tube, rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (WO 2010031713 A1, 93.4 mg, 0.20 mmol) were combined with ethyl 3-isocyanatobenzoate (Aldrich, 76.4 mg, 0.40 mmol) in CH₂Cl₂ (5.0 mL) to give a white suspension. The reaction mixture was heated at 120° C. for 15 min with CEM microwave reactor. The crude reaction mixture was concentrated in vacuo and the residue was purified by flash chromatography (silica gel, 12 g, 1% to 25% EtOAc in hexanes) to give a white amorphous (55.6 mg, 43.4%). HRMS (ES⁺) m/z Calcd for C₃₃H₂₉Cl₂F₂N₃O₄ [(M+Na)⁺]: 662.1395. Found: 662.1399.

### Example 32

### Preparation of rac-3-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid

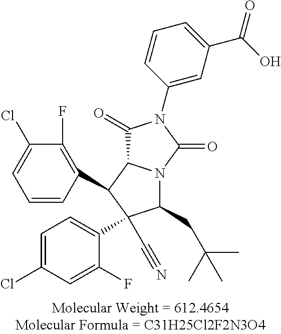

In a 10 mL pressure tube was placed rac-3-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid methyl ester (40.0 mg, 0.063 mmol) in MeCN, and AlI₃ (Aldrich, 50.9 mg, 0.125 mmol) was added in portions. The reaction mixture was heated to 130° C. for 15 min with CEM microwave reactor. The crude reaction mixture was poured into 25 mL ice-water and extracted with EtOAc. The organic layers were combined, washed with Na₂SO₃ (1×20 mL), H₂O (3×15 mL), and sat NaCl (1×15 mL). The crude reaction mixture was concentrated in vacuo. The crude material was purified by flash chromatography (silica gel, 12 g, 5% to 80% EtOAc in hexanes) to give a white amorphous (35.2 mg, 92.0%). HRMS (ES⁺) m/z Calcd for C₃₁H₂₅Cl₂F₂N₃O₄-[(M+Na)⁺]: 634.1082. Found: 634.1081.

### Example 33

### Preparation of chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-benzoic acid

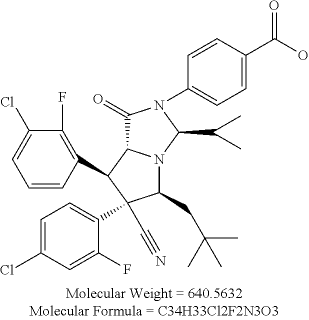

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester (WO 2010031713 A1, 75.0 mg, 125 umol) and isobutyraldehyde (45 mg, 0.624 mmol) were combined with AcOH (3 mL) and stirred at rt. Sodium triacetoxyborohydride (Fluka, 176 mg, 0.833 mmol) was added in two portions, 30 min apart and vigorously stirred for 3 h. The reaction mixture was diluted with 0.1 N NaOH and extracted with EtOAc. The organic layer was separated and concentrated under reduced pressure to afford crude product chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-benzoic acid methyl ester mix (82.1 mg, 100%) that was carried directly to the next step.

Compound chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-benzoic acid methyl ester mix (82.1 mg, 0.125 mmol) was dissolved in THF (3 mL) and methanol (1 mL), followed by 2N LiOH (0.5 mL) addition. The reaction mixture was stirred at room temperature for 5 hours, incomplete by LCMS, reaction mixture heated to 50° C. for 3 h, complete by LCMS. The reaction mixture was diluted with water and extracted with ethyl acetate (2×). The organic phase was separated, then concentrated under reduced pressure to afford crude material that was purified by RP-HPLC (35-95% acetonitrile/water) to gie an off-white solid (14.6 mg, 18.2%). HRMS (ES⁺) m/z Calcd for C₃₄H₃₃Cl₂F₂N₃O₃ [(M+H)⁺)⁺]: Calcd for: 640.1940. found: 640.1935.

### Example 34

### Preparation of rac-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-benzoic acid

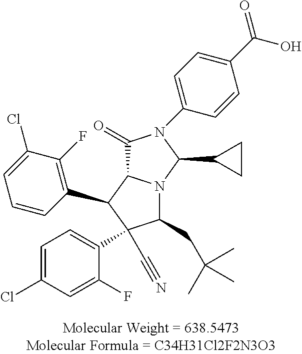

In a 10 mL round-bottomed flask, methyl 4-((2R,3S,4R,5S)-3-(3-chloro-2-fluorophenyl)-4-(4-chloro-2-fluorophenyl)-4-cyano-5-neopentylpyrrolidine-2-carboxamido)benzoate (WO 2010031713 A1, 25 mg, 0.042 mmol) and cyclopropanecarbaldehyde (Aldrich, 29.2 mg, 0.42 mmol) were combined with AcOH (1.00 ml) and CH₂Cl₂ (1 ml) to give a colorless solution. Sodium triacetoxyborohydride (Fluka, 176 mg, 0.833 mmol) was added in two portions 30 min apart. The reaction mixture was vigorously stirred for 5 h.

The reaction mixture was quenched with 2.0 N NaOH and extracted with EtOAc (3×20 mL).

The organic layers were dried over Na₂SO₄ and concentrated in vacuo.

The crude material was used in the next step without further purification.

In a 10 mL round-bottomed flask, the crude material obtained above was dissolved in a mixture of THF-MeOH-2.0 N KOH (1.8 mL-0.6 mL-0.6 mL). to give a colorless solution. The reaction mixture was stirred at rt overnight. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give 4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-benzoic acid (21.3 mg, 72.6%) and rac-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-benzoic acid as the by-product (5.3 mg, 18.1%). HRMS (ES⁺) m/z Calcd for C₃₄H₃₁Cl₂F₂N₃O₃-[(M+H)⁺]: 638.1784. Found: 638.1784.

### Example 35

### Preparation of rac-4-[(3S,5S,6R,7S,7aR)-7-(3-chloro-2-fluoro-phenyl)-6-(4-chloro-2-fluoro-phenyl)-6-cyano-3-cyclohexylmethyl-5-(2,2-dimethyl-propyl)-1-oxo-tetrahydro-pyrrolo[1,2-c]imidazol-2-yl]-benzoic acid

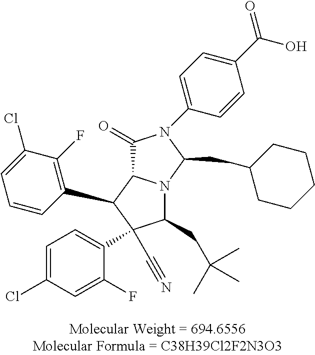

In a 10 mL round-bottomed flask, methyl 4-((2R,3S,4R,5S)-3-(3-chloro-2-fluorophenyl)-4-(4-chloro-2-fluorophenyl)-4-cyano-5-neopentylpyrrolidine-2-carboxamido)benzoate (WO 2010031713 A1, 30 mg, 0.050 mmol) and cyclohexylacetaldehyde (Aldrich, 63 mg, 0.42 mmol) were combined with AcOH (1.00 ml) and CH₂Cl₂ (1 ml) to give a colorless solution. Sodium triacetoxyborohydride (Fluka, 212 mg, 1.0 mmol) was added in two portions 30 min apart. The reaction mixture was vigorously stirred for 15 h.

The reaction mixture was quenched with 2.0 N NaOH and extracted with EtOAc (3×20 mL). The organic layers were dried over Na₂SO₄ and concentrated in vacuo.

The crude material was used in the next step without further purification.

In a 10 mL round-bottomed flask, the crude material obtained above was dissolved in a mixture of THF-MeOH-2.0 N KOH (1.8 mL-0.6 mL-0.6 mL). to give a colorless solution. The reaction mixture was stirred at rt overnight. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give 4-{[(2R,3S,4R,5S)-3-(3-chloro-2-fluoro-phenyl)-4-(4-chloro-2-fluoro-phenyl)-4-cyano-1-cyclohexylethyl-5-(2,2-dimethyl-propyl)-pyrrolidine-2-carbonyl]-amino}-benzoic acid (5.9 mg, 20.1%) and rac-4-[(3S,5S,6R,7S,7aR)-7-(3-chloro-2-fluoro-phenyl)-6-(4-chloro-2-fluoro-phenyl)-6-cyano-3-cyclohexylmethyl-5-(2,2-dimethyl-propyl)-1-oxo-tetrahydro-pyrrolo[1,2-c]imidazol-2-yl]-benzoic acid (4.3 mg, 14.7%) as the by-product. HRMS (ES⁺) m/z Calcd for C₃₈H₃₉Cl₂F₂N₃O₃ [(M+H)⁺]: 694.2410. Found: 694.2409.

### Example 36

### Preparation of chiral 4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-benzoic acid

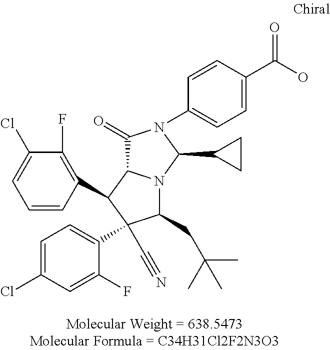

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester (WO 2010031713 A1, 75.0 mg, 119 umol) and cyclopropanecarboxaldehyde (93 mg, 1.33 mmol) were combined with AcOH (3 mL) and stirred at rt. Sodium triacetoxyborohydride (Fluka, 176 mg, 0.830 mmol) was added in two portions, 30 min apart and vigorously stirred for 3 h. The reaction mixture was diluted with 0.1 N NaOH and extracted with EtOAc. The organic layer was separated and concentrated under reduced pressure to afford crude product chiral 4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-benzoic acid methyl ester mix (81 mg, 100%) that was carried directly to the next step.

Compound chiral 4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-benzoic acid methyl ester mix (81 mg, 0.119 mmol) was dissolved in THF (3 mL) and methanol (1 mL), followed by 2N LiOH (0.5 mL) addition. The reaction mixture was stirred at room temperature for 12 hours, complete by LCMS. The reaction mixture was diluted with water and extracted with ethyl acetate (2×). The organic phase was separated, then concentrated under reduced pressure to give an off-white solid (15.2 mg, 38.8%). HRMS (ES⁺) m/z Calcd for C₃₄H₃₁Cl₂F₂N₃O₃ [(M+H)⁺]: calc: 668.1889. Found: 668.1891

### Example 37

Preparation of chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-2-methoxy-benzoic acid

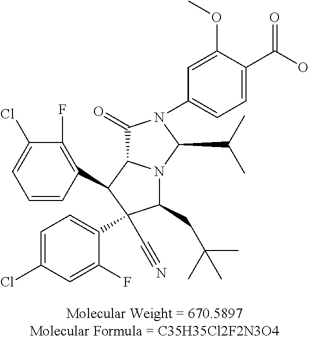

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester (WO 2010031713 A1, 75.0 mg, 119 umol) and isobutyraldehyde (42.9 mg, 0.595 mmol) were combined with AcOH (3 mL) and stirred at rt. Sodium triacetoxyborohydride (Fluka, 176 mg, 0.830 mmol) was added in two portions, 30 min apart and vigorously stirred for 3 h. Additional isobutyraldehyde (0.1 mL, 1.76 mmol) and sodium triacetoxyborohydride (100 mg, 0.47 mmol) were added and the mixture was stirred an additional 3 hours. The reaction mixture was diluted with 0.1 N NaOH and extracted with EtOAc. The organic layer was separated and concentrated under reduced pressure to afford crude product chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-2-methoxy-benzoic acid methyl ester mix (80 mg, 98%) that was carried directly to the next step. Compound chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-2-methoxy-benzoic acid methyl ester mix (80 mg, 0.116 mmol) was dissolved in THF (3 mL) and methanol (1 mL), followed by 2N LiOH (1 mL) addition. The reaction mixture was stirred at room temperature for 12 hours then was diluted with water and extracted with ethyl acetate (2×). The organic phase was separated, then concentrated under reduced pressure to afford crude mixture that was purified by RP-HPLC (30-95% acetonitrile/water) to give an off-white solid (30.1 mg, 44.9%). HRMS (ES⁺) m/z Calcd for C₃₅H₃₅Cl₂F₂N₃O₄ [(M+H)⁺]: calc: 670.2046. Found: 670.2041.

### Example 38

### Preparation of rac-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-3-methoxy-benzoic acid

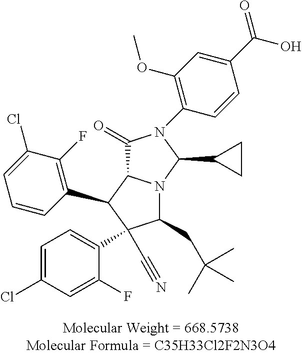

In a manner similar to Example 35, the solution of methyl 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (WO 2010031713 A1, 31.5 mg, 0.05 mmol) and cyclopropanecarboxaldehyde (Aldrich, 35.0 mg, 0.5 mmol) in AcOH (1.00 mL) and CH₂Cl₂ (1.00 mL) was treated with sodium triacetoxyborohydride (Fluka, 212 mg, 1.0 mmol) at rt for 15 h.

The crude material obtained above was hydrolyzed in a mixture of THF-MeOH-2.0 N KOH (1.8 mL-0.6 mL-0.6 mL). to give rac-rac-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-3-methoxy-benzoic acid (8.5 mg, 25.5%) as the by-product. HRMS (ES⁺) m/z Calcd for C₃₃H₃₁Cl₂F₂N₃O₄ [(M+H)]: 642.1733. Found: 642.1734.

### Example 39

Preparation of rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-methyl-1-oxo-tetrahydro-pyrrolo[1,2-c]imidazol-2-yl]-3-methoxy-benzoic acid

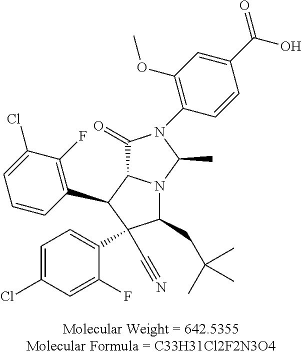

In a manner similar to Example 35, the solution of methyl 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (WO 2010031713 A1, 63.1 mg, 0.10 mmol) and acetaldehyde (Aldrich, 44.1 mg, 1.0 mmol) in AcOH (1.00 mL) and CH₂Cl₂ (1.00 mL) was treated with sodium triacetoxyborohydride (Fluka, 212 mg, 1.0 mmol) at rt for 15 h.

The crude material obtained above was hydrolyzed in a mixture of THF-MeOH-2.0 N KOH (1.8 mL-0.6 mL-0.6 mL) to give rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-methyl-1-oxo-tetrahydro-pyrrolo[1,2-c]imidazol-2-yl]-3-methoxy-benzoic acid (15.3 mg, 23.8%) as the by-product. HRMS (ES⁺) m/z Calcd for C₃₃H₃₁Cl₂F₂N₃O₄ [(M+H)⁺]. 642.1733. Found: 642.1734.

### Example 40

Preparation of rac 4-[(3S,5S,6R,7S,7aR)-3-butyl-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2c]imidazol-2-yl]-2-methoxy-benzoic acid

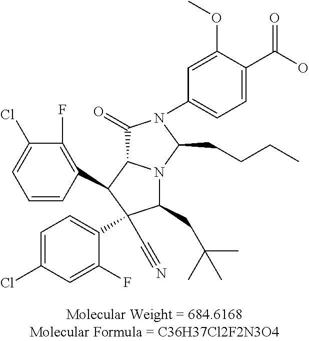

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester (WO 2010031713 A1, 230 mg, 397 umol) and valeraldehyde (0.1 mL, 81 mg, 0.94 mmol) were combined with AcOH (3 mL) and stirred at rt. Sodium triacetoxyborohydride (Fluka, 176 mg, 0.830 mmol) was added in two portions, 30 min apart and vigorously stirred for 3 h. The reaction mixture was diluted with 0.1 N NaOH and extracted with EtOAc and washed with water. The organic layer was separated and concentrated under reduced pressure to afford crude product 4-[(3S,5S,6R,7S,7aR)-3-butyl-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2c]imidazol-2-yl]-2-methoxy-benzoic acid methyl ester (0.23 g, 82.8%) that was carried directly to the next step.

Compound 4-[(3S,5S,6R,7S,7aR)-3-butyl-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2c]imidazol-2-yl]-2-methoxy-benzoic acid methyl ester mix (120 mg, 0.171 mmol) was dissolved in THF (3 mL) and methanol (1 mL), followed by 2N LiOH (1 mL) addition. The reaction mixture was stirred at room temperature for 14 hours at 25° C. The reaction mixture was diluted with water and extracted with ethyl acetate (2×). The organic phase was separated, then concentrated under reduced pressure to afford crude mixture that was purified by RP-HPLC (40-95% acetonitrile/water) to afford an off-white solid (11.3 mg, 19.3%). HRMS (ES⁺) m/z Calcd for C₃₆H₃₇Cl₂F₂N₃O₄ [(M+H)⁺]: 684.2202. found: 684.2204.

### Example 41

### Preparation of 4-[(3S,5S,6R,7S,7aR)-3-but-3-enyl-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-methoxy-benzoic acid

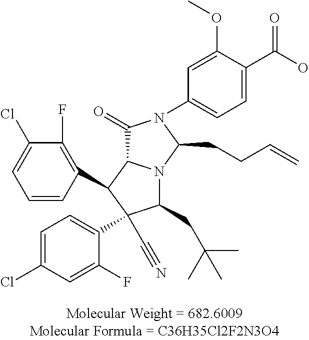

In a round-bottomed flask, rac 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester (WO 2010031713 A1, 110 mg, 174 mol) and 4-pentenal (0.1 mL, 85.2 mg, 1.01 mmol) were combined with AcOH (3 mL) and stirred at rt. Sodium triacetoxyborohydride (Fluka, 176 mg, 0.830 mmol) was added in two portions, 30 min apart and vigorously stirred for 3 h. The reaction mixture was diluted with 0.1 N NaOH and extracted with EtOAc and washed with water. The organic layer was separated and concentrated under reduced pressure to afford crude product 4-[(3S,5S,6R,7S,7aR)-3-but-3-enyl-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-methoxy-benzoic acid methyl ester (100 mg, 82.0%) that was carried directly to the next step.

Compound rac 4-[(3S,5S,6R,7S,7aR)-3-but-3-enyl-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-methoxy-benzoic acid methyl ester mix (120 mg, 0.171 mmol) was dissolved in THF (3 mL) and methanol (1 mL), followed by 2N LiOH (1 mL) addition. The reaction mixture was stirred at room temperature for 14 hours at 25° C. The reaction mixture was diluted with water and extracted with ethyl acetate (2×). The organic phase was separated, then concentrated under reduced pressure to afford crude mixture that was purified by RP-HPLC (40-95% acetonitrile/water) to give an off-white solid (19.7 mg, 33.6%). HRMS (ES⁺) m/z Calcd for C₃₆H₃₇Cl₂F₂N₃O₄ [(M+Na)¹⁺]: Calcd for: 704.1865. Found: 704.1862.

### Example 42

### Preparation of rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-3-methoxy-benzoic acid

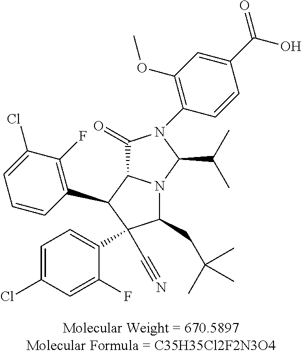

In a manner similar to Example 35, the solution of methyl 4-(2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (WO 2010031713 A1, 63.1 mg, 0.10 mmol) and isobutyraldehyde (Aldrich, 72.1 mg, 1.0 mmol) in AcOH (4.00 mL) and CH₂Cl₂ (4.0 mL) was treated with sodium triacetoxyborohydride (Fluka, 212 mg, 1.0 mmol) at rt for 15 h.

The crude material obtained above was hydrolyzed in a mixture of THF-MeOH-2.0 N KOH (1.8 mL-0.6 mL-0.6 mL) to give rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-3-methoxy-benzoic acid (27.6 mg, 41.2%). HRMS (ES⁺) m/z Calcd for C₃₅H₃₅Cl₂F₂N₃O₄ [(M+H)⁺]: 670.2045. Found: 670.2064.

### Example 43

### Preparation of chiral-4-[(3 S,5S,6R,7S,7aR)-7-(3-chloro-2-fluoro-phenyl)-6-(4-chloro-2-fluoro-phenyl)-6-cyano-5-(2,2-dimethyl-propyl)-3-methyl-1-oxo-tetrahydro-pyrrolo[1,2-c]imidazol-2-yl]-3-methoxy-benzoic acid

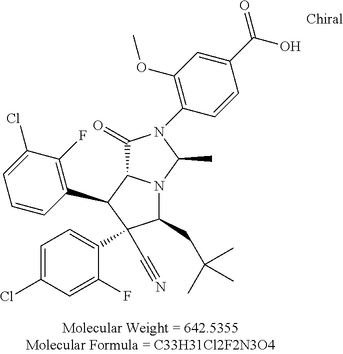

In a 10 mL pressure tube, the solution of chiral-methyl 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (WO 2010031713 A1, 31.5 mg, 0.05 mmol) and acetaldehyde (Aldrich, 44.1 mg, 1.0 mmol) in AcOH (1.0 mL) and CH₂Cl₂ (1.0 mL) was stirred at 50° C. overnight. The reaction mixture was quenched with 2.0 N NaOH and extracted with EtOAc (3×20 mL). The organic layers were dried over Na₂SO₄ and concentrated in vacuo. The crude material was purified by flash chromatography (silica gel, 4 g, 1% to 20% EtOAc in Hexanes) to give chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-methyl-1-oxo-tetrahydro-pyrrolo[1,2-c]imidazol-2-yl]-3-methoxy-benzoic acid methyl ester (30.4 mg, 92.6%).

In a 10 mL round-bottomed flask, chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-methyl-1-oxo-tetrahydro-pyrrolo[1,2-c]imidazol-2-yl]-3-methoxy-benzoic acid methyl ester (28.0 mg, 0.043 mmol) obtained above was dissolved in a mixture of THF-MeOH-2.0 N KOH (1.8 mL-0.6 mL-0.6 mL) to give a colorless solution. The reaction mixture was stirred at rt overnight. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give a white powder (25.3 mg, 87.4%).

HRMS (ES⁺) m/z Calcd for C₃₃H₃₁Cl₂F₂N₃O₄ [(M+H)⁺]: 642.1733. Found: 642.1730.

### Example 44

### Preparation of rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isobutyl-1-oxo-tetrahydro-pyrrolo[1,2-c]imidazol-2-yl]-3-methoxy-benzoic acid

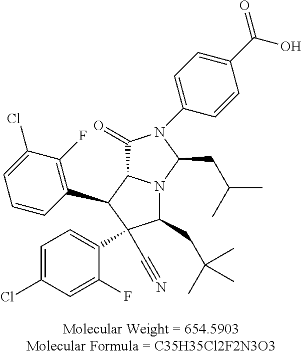

In a 10 mL pressure tube, the solution of methyl 4-((2R,3S,4R,5S)-3-(3-chloro-2-fluorophenyl)-4-(4-chloro-2-fluorophenyl)-4-cyano-5-neopentylpyrrolidine-2-carboxamido)-benzoate (WO 2010031713 A1, 30.0 mg, 0.05 mmol) and 3-methylbutanal (Aldrich, 43.0 mg, 1.0 mmol) in AcOH (1.0 mL) and CH₂Cl₂ (1.0 mL) was heated in the CEM microwave reactor at 120° C. for 15 minutes. The reaction mixture was quenched with 2.0 N NaOH and extracted with EtOAc (3×20 mL). The organic layers were dried over Na₂SO₄ and concentrated in vacuo. The crude material was used in the next step without further purification

In a 10 mL round-bottomed flask, the crude material obtained above was dissolved in a mixture of THF-MeOH-2.0 N KOH (1.8 mL-0.6 mL-0.6 mL) to give a colorless solution. The reaction mixture was stirred at rt overnight. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give a white powder (28.4 mg, 82.2%). HRMS (ES⁺) m/z Calcd for C₃₅H₃₅Cl₂F₂N₃O₃ [(M+H)⁺]: 654.2097. Found: 654.2099.

### Example 45

### Preparation of chiral-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-3-methoxy-benzoic acid

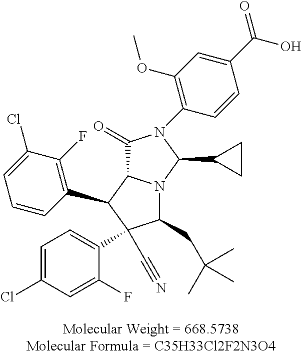

In a 10 mL pressure tube, the solution of chiral-methyl 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (WO 2010031713 A1, 31.5 mg, 0.05 mmol) and cyclopropanecarbaldehyde (Aldrich, 70.1 mg, 1.0 mmol) in AcOH (1.0 mL) and CH₂Cl₂ (1.0 mL) was stirred at 50° C. overnight and then 70° C. for 5 hrs. The reaction mixture was quenched with 2.0 N NaOH and extracted with EtOAc (3×20 mL). The organic layers were dried over Na₂SO₄ and concentrated in vacuo. The crude material was purified by flash chromatography (silica gel, 4 g, 1% to 20% EtOAc in Hexanes) to give chiral-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-3-methoxy-benzoic acid methyl ester (21.9 mg, 64.2%).

In a 10 mL round-bottomed flask, chiral-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-3-methoxy-benzoic acid methyl ester (20.0 mg, 0.029 mmol) obtained above was dissolved in a mixture of THF-MeOH-2.0 N KOH (1.8 mL-0.6 mL-0.6 mL) to give a colorless solution. The reaction mixture was stirred at rt overnight. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give a white powder (15.3 mg, 66.7%).

HRMS (ES⁺) m/z Calcd for C₃₅H₃₃Cl₂F₂N₃O₄ [(M+H)^{+]}: 668.1889. Found: 663.1891.

### Example 46

### Preparation of chiral-4-[(3S,5S,6R,7S,7aR)-7-(3-chloro-2-fluoro-phenyl)-6-(4-chloro-2-fluoro-phenyl)-6-cyano-3-cyclohexylmethyl-5-(2,2-dimethyl-propyl)-1-oxo-tetrahydro-pyrrolo[1,2-c]imidazol-2-yl]-3-methoxy-benzoic acid

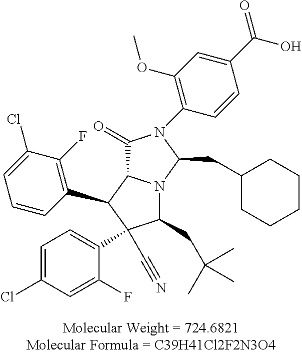

In a 10 mL pressure tube, the solution of chiral-methyl 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (WO 2010031713 A1, 31.5 mg, 0.05 mmol) and 3-cyclohexylacetaldehyde (Aldrich, 63.1 mg, 1.0 mmol) in AcOH (1.0 mL) and CH₂Cl₂ (1.0 mL) was stirred at 50° C. overnight and then 70° C. for 5 hrs. The reaction mixture was quenched with 2.0 N NaOH and extracted with EtOAc (3×20 mL). The organic layers were dried over Na₂SO₄ and concentrated in vacuo. The crude material was purified by flash chromatography (silica gel, 4 g, 1% to 20% EtOAc in Hexanes) to give chiral-4-[(3S,5S,6R,7S,7aR)-7-(3-chloro-2-fluoro-phenyl)-6-(4-chloro-2-fluoro-phenyl)-6-cyano-3-cyclohexylmethyl-5-(2,2-dimethyl-propyl)-1-oxo-tetrahydro-pyrrolo[1,2-c]imidazol-2-yl]-3-methoxy-benzoic acid methyl ester (33.4 mg, 90.4%).

In a 10 mL round-bottomed flask, chiral-4-[(3S,5S,6R,7S,7aR)-7-(3-chloro-2-fluoro-phenyl)-6-(4-chloro-2-fluoro-phenyl)-6-cyano-3-cyclohexylmethyl-5-(2,2-dimethyl-propyl)-1-oxo-tetrahydro-pyrrolo[1,2-c]imidazol-2-yl]-3-methoxy-benzoic acid methyl ester (30.0 mg, 0.041 mmol) obtained above was dissolved in a mixture of THF-MeOH-2.0 N KOH (1.8 mL-0.6 mL-0.6 mL) to give a colorless solution. The reaction mixture was stirred at rt overnight. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give chiral-4-[(3S,5S,6R,7S,7aR)-7-(3-chloro-2-fluoro-phenyl)-6-(4-chloro-2-fluoro-phenyl)-6-cyano-3-cyclohexylmethyl-5-(2,2-dimethyl-propyl)-1-oxo-tetrahydro-pyrrolo[1,2-c]imidazol-2-yl]-3-methoxy-benzoic acid hydrogen chloride salt (22.7 mg, 73.4%) as white powder.

HRMS (ES⁺) m/z Calcd for C₃₉H₄₁Cl₂F₂N₃O₄ [(M+H)⁺]: 724.2515. Found: 724.2515.

### Example 47

### Preparation of chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-3-methoxy-benzoic acid

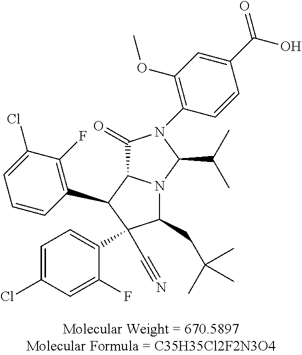

In a 10 mL pressure tube, the solution of chiral-methyl 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (WO 2010031713 A1, 31.5 mg, 0.05 mmol) and isobutyraldehyde (Aldrich, 36.1 mg, 1.0 mmol) in AcOH (1.0 mL) and CH₂Cl₂ (1.0 mL) was stirred at 50° C. overnight and then 70° C. for 5 hrs. The reaction mixture was quenched with 2.0 N NaOH and extracted with EtOAc (3×20 mL). The organic layers were dried over Na₂SO₄ and concentrated in vacuo. The crude material was purified by flash chromatography (silica gel, 4 g, 1% to 20% EtOAc in Hexanes) to give chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-3-methoxy-benzoic acid methyl ester (32.6 mg, 95.2%).

In a 10 mL round-bottomed flask, chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-3-methoxy-benzoic acid methyl ester (31.0 mg, 0.045 mmol) obtained above was dissolved in a mixture of THF-MeOH-2.0 N KOH (1.8 mL-0.6 mL-0.6 mL) to give a colorless solution. The reaction mixture was stirred at rt overnight. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-3-methoxy-benzoic acid trifluoroacetic acid salt (18.8 mg, 53.0%) as white powder.

HRMS (ES⁺) m/z Calcd C₃₅H₃₅Cl₂F₂N₃O₄ [(M+H)⁺]: 670.2046. Found: 670.2045.

### Example 48

Preparation of chiral-4-[(3S,5S,6R,7S,7aR)-7-(3-chloro-2-fluoro-phenyl)-6-(4-chloro-2-fluoro-phenyl)-6-cyano-3-cyclobutyl-5-(2,2-dimethyl-propyl)-1-oxo-tetrahydro-pyrrolo[1,2-c]imidazol-2-yl]-3-methoxy-benzoic acid

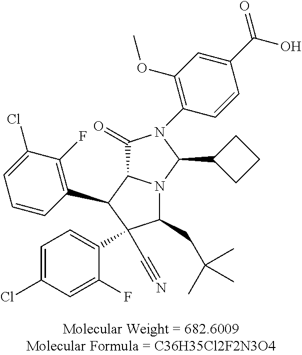

In a 10 mL pressure tube, the solution of chiral-methyl 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (WO 2010031713 A1, 31.5 mg, 0.05 mmol) and cyclobutanecarbaldehyde (Aldrich, 42.1 mg, 1.0 mmol) in AcOH (1.0 mL) and CH₂Cl₂ (1.0 mL) was stirred at 50° C. overnight and then 70° C. for 5 hrs. The reaction mixture was quenched with 2.0 N NaOH and extracted with EtOAc (3×20 mL). The organic layers were dried over Na₂SO₄ and concentrated in vacuo. The crude material was purified by flash chromatography (silica gel, 4 g, 1% to 20% EtOAc in Hexanes) to give chiral-4-[(3S,5S,6R,7S,7aR)-7-(3-chloro-2-fluoro-phenyl)-6-(4-chloro-2-fluoro-phenyl)-6-cyano-3-cyclobutyl-5-(2,2-dimethyl-propyl)-1-oxo-tetrahydro-pyrrolo[1,2-c]imidazol-2-yl]-3-methoxy-benzoic acid methyl ester (33.0 mg, 94.7%).

In a 10 mL round-bottomed flask, chiral-4-[(3S,5S,6R,7S,7aR)-7-(3-chloro-2-fluoro-phenyl)-6-(4-chloro-2-fluoro-phenyl)-6-cyano-3-cyclobutyl-5-(2,2-dimethyl-propyl)-1-oxo-tetrahydro-pyrrolo[1,2-c]imidazol-2-yl]-3-methoxy-benzoic acid methyl ester (33.0 mg, 0.047 mmol) obtained above was dissolved in a mixture of THF-MeOH-2.0 N KOH (1.8 mL-0.6 mL-0.6 mL) to give a colorless solution. The reaction mixture was stirred at rt overnight. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give chiral-4-[(3S,5S,6R,7S,7aR)-7-(3-chloro-2-fluoro-phenyl)-6-(4-chloro-2-fluoro-phenyl)-6-cyano-3-cyclobutyl-5-(2,2-dimethyl-propyl)-1-oxo-tetrahydro-pyrrolo[1,2-c]imidazol-2-yl]-3-methoxy-benzoic acid trifluoroacetic acid salt (14.6 mg, 38.7%) as white powder.

HRMS (ES⁺) m/z Calcd for C₃₆H₃₅Cl₂F₂N₃O₄ [(M+H)⁺]: 682.2046. Found: 682.2042

### Example 49

### Preparation of rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-hydroxymethyl-1-oxo-tetrahydro-pyrrolo[1,2-c]imidazol-2-yl]-benzoic acid

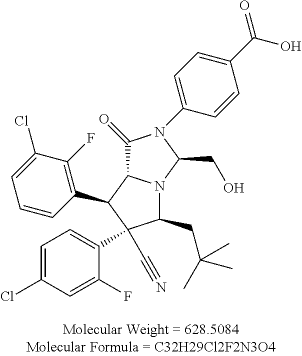

In a 10 mL pressure tube, the solution of chiral-methyl 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (WO 2010031713 A1, 30.0 mg, 0.05 mmol) and 2-(tert-butyldimethylsilyloxy)acetaldehyde (Aldrich, 43.5 mg, 0.25 mmol) in AcOH (1.0 mL) and CH₂Cl₂ (1.0 mL) was stirred at 75° C. overnight. The reaction mixture was quenched with 2.0 N NaOH and extracted with EtOAc (3×20 mL). The organic layers were dried over Na₂SO₄ and concentrated in vacuo. The crude material was purified by flash chromatography (silica gel, 4 g, 1% to 20% EtOAc in Hexanes) to give rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-hydroxymethyl-1-oxo-tetrahydro-pyrrolo[1,2-c]imidazol-2-yl]-benzoic acid methyl ester (8.3.0 mg, 25.9%).

In a 10 mL round-bottomed flask, rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-hydroxymethyl-1-oxo-tetrahydro-pyrrolo[1,2-c]imidazol-2-yl]-benzoic acid methyl ester (7.0 mg, 0.011 mmol) obtained above was dissolved in a mixture of THF-MeOH-2.0 N KOH (1.8 mL-0.6 mL-0.6 mL) to give a colorless solution. The reaction mixture was stirred at rt overnight. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative R^{P}-HPLC to give rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-hydroxymethyl-1-oxo-tetrahydro-pyrrolo[1,2-c]imidazol-2-yl]-benzoic acid hydrogen chloride salt (6.3 mg, 87.0%) as white powder.

HRMS (ES⁺) m/z Calcd for C₃₂H₂₉Cl₂F₂N₃O₄⁺H [(M+H)]: 628.1576. Found: 628.1575.

### Example 50

### Preparation of rac 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-ethyl-1-oxo-tetrahydro-pyrrolo[1,2c]imidazol-2-yl]-2-methoxy-benzoic acid

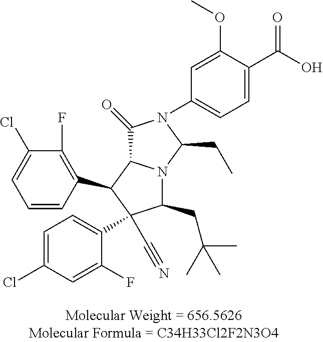

In a round-bottomed flask, rac 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester (WO 2010031713 A1, 100 mg, 159 umol) and propionaldehyde (0.1 mL, 80.5 mg, 1.01 mmol) were combined with AcOH (3 mL) and stirred at rt. Sodium triacetoxyborohydride (Fluka, 176 mg, 0.830 mmol) was added in two portions, 30 min apart and vigorously stirred for 3 h. LCMS showed product. The reaction mixture was diluted with 0.1 N NaOH and extracted with EtOAc and washed with water. The organic layer was separated and concentrated under reduced pressure to afford crude product that was purified by RP-HPLC (40-95% acetonitrile/water) to afford rac 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-ethyl-1-oxo-tetrahydro-pyrrolo[1,2c]imidazol-2-yl]-2-methoxy-benzoic acid methyl ester (32.1 mg, 30.6%) that was carried directly to the next step.

Compound rac 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-ethyl-1-oxo-tetrahydro-pyrrolo[1,2c]imidazol-2-yl]-2-methoxy-benzoic acid methyl ester (32 mg, 47.7 mol) was dissolved in THF (1.5 mL) and methanol (0.5 mL), followed by 2N LiOH (0.5 mL) addition. The reaction mixture was stirred at room temperature for 4 hours at 25° C. LCMS showed product. The reaction mixture was diluted with water and extracted with ethyl acetate (2×). The organic phase was separated, then concentrated under reduced pressure to afford crude mixture that was purified by RP-HPLC (40-95% acetonitrile/water) to afford an off-white solid (23.4 mg, 74.7%). HRMS (ES⁺) m/z Calcd for C₃₄H₃₃Cl₂F₂N₃O₄ [(M+)¹⁺]: Calcd for: 656.1889. Found: 656.1885.

### Example 51

### Preparation of rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-benzoic and

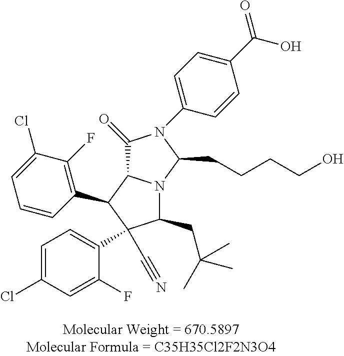

In a 10 mL pressure tube, to the solution of chiral-methyl 4-((2R,3S,4R,5S)-3-(3-chloro-2-fluorophenyl)-4-(4-chloro-2-fluorophenyl)-4-cyano-5-neopentylpyrrolidine-2-carboxamido)-benzoate (WO 2010031713 A1, 30.0 mg, 0.05 mmol) and 5-hydroxypentanal (Aldrich, 51.0 mg, 0.5 mmol) in AcOH (1.0 mL) and CH₂Cl₂ (1.0 mL), sodium triacetoxyborohydride (Fluka, 212 mg, 1.0 mmol) was added in two portions 30 min apart. The reaction mixture was vigorously stirred at rt for 15 h. The reaction mixture was quenched with 2.0 N NaOH and extracted with EtOAc (3×20 mL). The organic layers were dried over Na₂SO₄ and concentrated in vacuo. The crude material was used in the next step without further purification.

In a 10 mL round-bottomed flask, the crude material obtained above was dissolved in a mixture of THF-MeOH-2.0 N KOH (1.8 mL-0.6 mL-0.6 mL) to give a colorless solution. The reaction mixture was stirred at rt for 5 hrs. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-benzoic acid trifluoroacetic acid salt (6.5 mg, 16.6%) as by-product. HRMS (ES⁺) m/z Calcd for C₃₅H₃₅Cl₂F₂N₃O₄ [(M+H)⁺]: 670.2046. Found: 670.2046.

### Example 52

### Preparation of chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-3-methoxy-benzoic acid

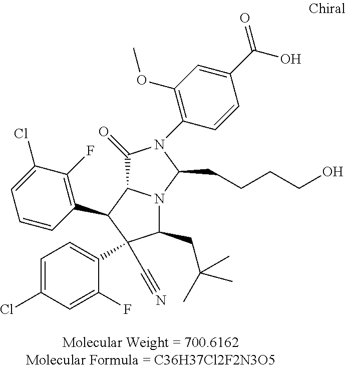

In a 10 mL pressure tube, to the solution of chiral-methyl 4-((2R,3S,4R,5S)-3-(3-chloro-2-fluorophenyl)-4-(4-chloro-2-fluorophenyl)-4-cyano-5-neopentylpyrrolidine-2-carboxamido)-benzoate (WO 2010031713 A1, 63.0 mg, 0.10 mmol) and 5-hydroxypentanal (Aldrich, 102.0 mg, 1.0 mmol) in AcOH (1.0 mL) and CH₂Cl₂ (1.0 mL), sodium triacetoxyborohydride (Fluka, 424 mg, 2.0 mmol) was added in two portions 30 min apart. The reaction mixture was vigorously stirred at rt for 15 h. The reaction mixture was quenched with 2.0 N NaOH and extracted with EtOAc (3×20 mL). The organic layers were dried over Na₂SO₄ and concentrated in vacuo. The crude material was used in the next step without further purification.

In a 10 mL round-bottomed flask, the crude material obtained above was dissolved in a mixture of THF-MeOH-2.0 N KOH (1.8 mL-0.6 mL-0.6 mL) to give a colorless solution. The reaction mixture was stirred at rt for 5 hrs. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-3-methoxy-benzoic acid trifluoroacetic acid salt (14.6 mg, 17.9%) as by-product. HRMS (ES⁺) m/z Calcd for C₃₆H₃₇Cl₂F₂N₃O₅ [(M+H)⁺]: 700.2151. Found: 700.2152.

### Example 53

### Preparation of chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-hydroxymethyl-1-oxo-tetrahydro-pyrrolo[1,2-c]imidazol-2-yl]-3-methoxy-benzoic acid

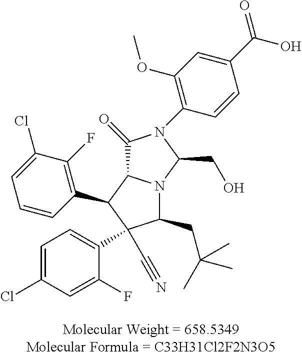

To a 10 mL microwave vial was added 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoic acid (WO 2010031713 A1, 61 mg, 98.9 μmol), 2-(tert-butyldimethylsilyloxy)acetaldehyde (172 mg, 989 μmol) in AcOH (1.00 ml) and DCM (1 ml) to give a colorless solution. The vial was capped and heated with the CEM microwave reactor at 120° C. for 45 min. More aldehyde added and the reaction mixture was heated for another 30 min at 120° C.

To the mixture was added 1.0 mL H2O and 2.0 mL AcOH, the reaction mixture was heated with the CEM microwave reactor at 120° C. for 30 min. The reaction mixture was diluted water and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give a white powder (15.8 mg, 24.2%).

HRMS (ES⁺) m/z Calcd for C₃₃H₃₁Cl₂F₂N₃O₅ [(M+H)⁺]: 658.1682. Found: 658.1678.

### Example 54

Preparation of rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-1-oxo-3-(tetrahydro-pyran-4-ylmethyl)-tetrahydro-pyrrolo[1,2-c]imidazol-2-yl]-benzoic acid

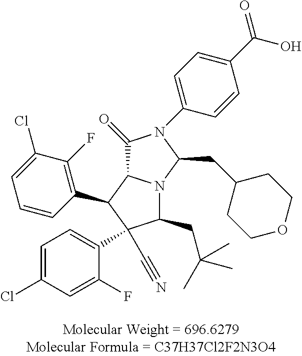

To a 10 mL microwave vial was added rac-4-((2R,3S,4R,5S)-3-(3-chloro-2-fluorophenyl)-4-(4-chloro-2-fluorophenyl)-4-cyano-5-neopentylpyrrolidine-2-carboxamido)benzoic acid (WO 2010031713 A1, 25 mg, 42.6 μmol), 2-(tetrahydro-2H-pyran-4-yl)acetaldehyde (54.6 mg, 426 μmol) and in AcOH (1.00 mL) and 1,2-dichloroethane (1.00 mL). The vial was capped and heated in the microwave at 120° C. for 60 min. The reaction mixture was diluted water and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give a white powder (18.9 mg, 63.6%). HRMS (ES⁺) m/z Calcd for C₃₇H₃₇Cl₂F₂N₃O₄ [(M+H)⁺]: 629.2202. Found: 629.2200.

### Example 55

Preparation of chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-1-oxo-3-(tetrahydro-pyran-4-ylmethyl)-tetrahydro-pyrrolo[1,2-c]imidazol-2-yl]-2-methoxy-benzoic acid

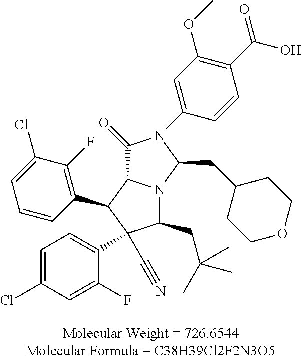

To a 10 mL microwave vial was added chiral methyl 4-((2R,3S,4R,5S)-3-(3-chloro-2-fluorophenyl)-4-(4-chloro-2-fluorophenyl)-4-cyano-5-neopentylpyrrolidine-2-carboxamido)-2-methoxybenzoate (63.0 mg, 99.9 μmol), 2-(tetrahydro-2H-pyran-4-yl)acetaldehyde (128 mg, 0.999 mmol) and in AcOH (1.00 ml) and 1,2-dichloroethane (1.00 ml). The vial was capped and heated in the microwave at 140° C. for 2 h.

The reaction mixture was poured into 20 mL H₂O and extracted with dichloromethane (3×50 mL). The organic layers were dried over Na₂SO₄ and concentrated in vacuo. The residue was used in the next step without further purification.

In a 10 mL round-bottomed flask, the crude mixture of methyl 4-((3S,5S,6R,7S,7aR)-7-(3-chloro-2-fluorophenyl)-6-(4-chloro-2-fluorophenyl)-6-cyano-5-neopentyl-1-oxo-3-((tetrahydro-2H-pyran-4-yl)methyl)-1H-pyrrolo[1,2-c]imidazol-2(3H,5H,6H,7H,7aH)-yl)-2-methoxybenzoate (74.1 mg, 100 μmol) were dissolved in the mixture of THF (1.80 mL)-MeOH (0.6 mL)-KOH (0.6 mL). The reaction mixture was heated to rt and stirred for 5 hrs. The reaction mixture was quenched with 0.5 mL 1 M HCl and extracted with EtOAc (3×25 mL). The organic layers were combined, washed with H₂O (1×10 mL), sat NaCl (1×10 mL), and dried over Na₂SO₄ and concentrated in vacuo.

The crude material was purified by preparative RP-HPLC to give a white amorphous (40.3 mg, 55.4%). HRMS (ES⁺) m/z Calcd for C₃₈H₃₉Cl₂F₂N₃O₅ [(M+H)⁺]: 726.1576. Found: 726.1575.

### Example 56

### Preparation of chiral-4-[(3 S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-2-methoxy-benzoic acid

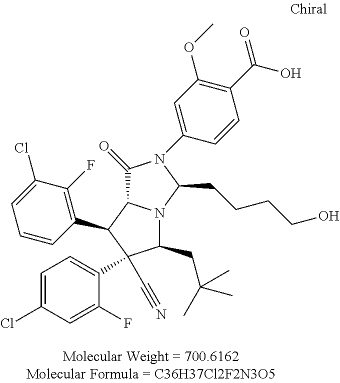

In a 10 mL microwave vial, chiral methyl 4-((2R,3S,4R,5S)-3-(3-chloro-2-fluorophenyl)-4-(4-chloro-2-fluorophenyl)-4-cyano-5-neopentylpyrrolidine-2-carboxamido)-2-methoxybenzoate (WO 2010031713 A1, 63.0 mg, 99.9 μmol) and 6-hydroxypentanal (102 mg, 0.999 mmol) were combined and suspended in a mixture of AcOH (1.00 ml) and 1,2-dichloroethane (1.00 ml). To the white suspension, sodium triacetoxyborohydride (Fluka, 424 mg, 2.0 mmol) was added in two portions 30 min apart. The reaction mixture was vigorously stirred at rt for 15 h. The reaction mixture was quenched with 2.0 N NaOH and extracted with EtOAc (3×20 mL). The organic layers were dried over Na₂SO₄ and concentrated in vacuo. The crude material was used in the next step without further purification.

In a 10 mL round-bottomed flask, the crude material obtained above was dissolved in a mixture of THF-MeOH-2.0 N KOH (1.8 mL-0.6 mL-0.6 mL) to give a colorless solution. The reaction mixture was stirred at rt for 5 hrs. The reaction mixture was quenched with 0.5 mL 1.0 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-2-methoxy-benzoic acid (9.8 mg, 14.0%) as py-product. HRMS (ES⁺) m/z Calcd for C₃₆H₃₇Cl₂F₂N₃O₅ (M+H)^{+]}: 700.2151. Found: 700.2149.

### Example 57

### Preparation of intermediate [3,3-dimethyl-but-(E)-ylideneamino]-acetic acid tert-butyl ester

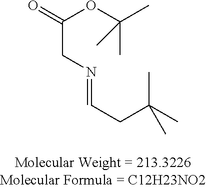

A mixture of glycine tert-butyl ester (Alfa (2.71 g, 20.0 mmol) and 3,3-dimethyl-butyraldehyde (Alfa) (2.21 g, 21.0 mmol) in CH₂Cl₂ (50 mL) was stirred at rt overnight. The reaction mixture was concentrated and the residue was dried in vacuo to give [3,3-dimethyl-but-(E)-ylideneamino]-acetic acid tert-butyl ester

(4.29 g, 100%) as colorless oil which was used in the next step without further purification.

### Example 58

### Preparation of intermediate (Z)-3-(3-chloro-2-fluoro-phenyl)-2-(4-chloro-2-fluoro-phenyl)-acrylonitrile

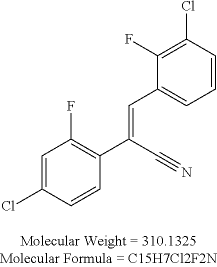

To a solution of 4-chloro-2-fluoro-phenylacetonitrile (Oakwood) (5 g, 30 mmol) and 3-chloro-2-fluorobenzaldehyde (Oakwood) (5 g, 32 mmol) in methanol (200 mL) was slowly added a methanolic solution (Aldrich, 25 wt. %) of sodium methoxide (21 mL, 92 mmol). The reaction mixture was heated and stirred at 45° C. for 5 h. The mixture became cloudy, and was cooled to room temperature and filtered. The white precipitate was washed with water, cold methanol, and then dried in vacuo to give the first batch of desired product. The filtrate was concentrated, diluted with water, neutralized by aqueous HCl solution to “pH” 7, then extracted with ethyl acetate. The organic layer was separated, dried over MgSO₄, and concentrated. The residue was purified by chromatography (EtOAc:hexanes=1:20, then 1:10) to give the second batch of the desired product. The two batches were combined to give (Z)-3-(3-chloro-2-fluoro-phenyl)-2-(4-chloro-2-fluoro-phenyl)-acrylonitrile as a white powder (9 g, 97%).

### Example 59

### Preparation of intermediate 2-((S)-2,2-dimethyl-[1,3]dioxolan-4-yl)-ethylamine

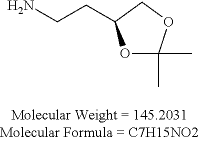

Step A. To a solution of (4S)-(+)-4-(2-hydroxyethyl)-2,2-dimethyl-1,3-dioxolane (Aldrich) (21.1 g, 0.14 mol) and triethylamine (40 mL, 0.28 mol) in dichloromethane (250 mL) at 0° C. was added methanesulfonyl chloride (13.4 mL, 0.17 mol) dropwise. The reaction mixture was stirred at 0° C. for 1.5 h, then water was added. The organic layer was separated, washed with water, brine, dried over MgSO₄, concentrated to give methanesulfonic acid 2-((S)-2,2-dimethyl-[1,3]dioxolan-4-yl)-ethyl ester as a yellow oil (31.7 g, 98%).

Step B. To a solution of methanesulfonic acid 2-((S)-2,2-dimethyl-[1,3]dioxolan-4-yl)-ethyl ester (31.7 g, 0.14 mol) in N,N-dimethylformamide (200 mL) was added NaN₃ (46 g, 0.71 mol). The reaction mixture was stirred at room temperature for 70 h. Then the mixture was partitioned between ethyl acetate and water. The organic layer was separated, washed with water, brine several times, dried over MgSO₄, concentrated to give (S)-4-(2-azido-ethyl)-2,2-dimethyl-[1,3]dioxolane as a yellow oil (21.3 g, 88%).

Step C. A suspension of (S)-4-(2-azido-ethyl)-2,2-dimethyl-[1,3]dioxolane as a yellow oil (18.7 g, 0.11 mol) and PtO₂ (2.5 g) in ethyl acetate (100 mL) was vigorously shaken in a Parr under atmosphere of H₂ (50 psi) for 18 h. The mixture was filtered through a short pad of celite. The filtrate was concentrated to give 2-((S)-2,2-dimethyl-[1,3]dioxolan-4-yl)-ethylamine as a colorless oil (14 g, 88%).

### Example 60

### Preparation of intermediate rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid tert-butyl ester

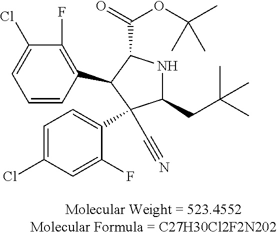

To a solution of [3,3-dimethyl-but-(E)-ylideneamino]-acetic acid tert-butyl ester (2.3 g, 11 mmol) and (Z)-3-(3-chloro-2-fluoro-phenyl)-2-(4-chloro-2-fluoro-phenyl)-acrylonitrile (2.5 g, 8 mmol) in dichloromethane (200 mL) were added triethylamine (2.9 g, 29 mmol) and AgF (0.7 g, 5.5 mmol) in one portion. The mixture was stirred at room temperature for 18 h. The mixture was concentrated. The residue was partitioned between ethyl acetate and brine. The organic layer was separated, dried over Na₂SO₄, and concentrated. The residue was dissolved into tert-butanol (10 mL), and DBU (10 mL) was added. The mixture was heated at 100° C. for 2 h, then cooled to room temperature, and concentrated. The residue was partitioned between ethyl acetate and water. The organic layer were separated, dried over MgSO₄, and concentrated. The residue was purified by chromatography (EtOAc:hexanes=1:10, 1:5) to give rac-(2R,3S,4R,5S)-3-(3-Chloro-2-fluoro-phenyl)-4-(4-chloro-2-fluoro-phenyl)-4-cyano-5-(2,2-dimethyl-propyl)-pyrrolidine-2-carboxylic acid tert-butyl ester as a white foam (3.6 g, 77%).

### Example 61

### Preparation of intermediate rac-(2R,3S,4R,5S)-1-[2-(tert-butyl-dimethyl-silanyloxy)-ethyl]-3-(3-chloro-2-fluoro-phenyl)-4-(4-chloro-2-fluoro-phenyl)-4-cyano-5-(2,2-dimethyl-propyl)-pyrrolidine-2-carboxylic acid tert-butyl ester

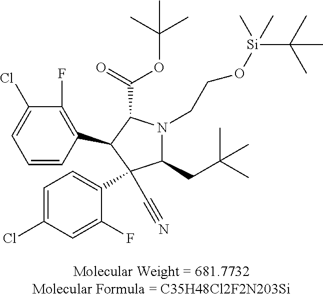

To the solution of rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid tert-butyl ester (0.33 g, 0.64 mmol) in dichloromethane (20 mL) was added (tert-butyldimethylsilyloxy)acetaldehyde (Aldrich) (1.7 g, 9.8 mmol) and sodium triacetoxyborohydride (Aldrich) (1.5 g, 7.1 mmol) sequentially. The reaction mixture was stirred at room temperature for 48 h. The mixture was concentrated, and the residue was partitioned between ethyl acetate and brine. The organic layer was separated, dried over MgSO₄, and concentrated. The residue was purified by chromatography (5% EtOAc in hexanes) to give rac-(2R,3S,4R,5S)-1-[2-(tert-butyl-dimethyl-silanyloxy)-ethyl]-3-(3-chloro-2-fluoro-phenyl)-4-(4-chloro-2-fluoro-phenyl)-4-cyano-5-(2,2-dimethyl-propyl)-pyrrolidine-2-carboxylic acid tert-butyl ester as a white gum (0.33 g, 77%).

### Example 62

Preparation of intermediate rac-(2R,3S,4R,5S)-1-[2-(tert-butyl-dimethyl-silanyloxy)-ethyl]-3-(3-chloro-2-fluoro-phenyl)-4-(4-chloro-2-fluoro-phenyl)-4-cyano-5-(2,2-dimethyl-propyl)-pyrrolidine-2-carboxylic acid trifluoroacetic acid

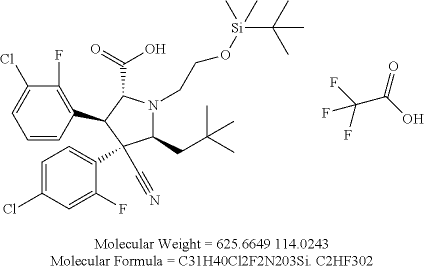

To a solution of rac-(2R,3S,4R,5S)-1-[2-(tert-butyl-dimethyl-silanyloxy)-ethyl]-3-(3-chloro-2-fluoro-phenyl)-4-(4-chloro-2-fluoro-phenyl)-4-cyano-5-(2,2-dimethyl-propyl)-pyrrolidine-2-carboxylic acid tert-butyl ester (0.33 g, 0.48 mmol) in dichloromethane (30 mL) was added trifluoroacetic acid (10 mL). The reaction mixture was stirred at room temperature for 18 h, and concentrated to give crude rac-(2R,3S,4R,5S)-1-[2-(tert-butyl-dimethyl-silanyloxy)-ethyl]-3-(3-chloro-2-fluoro-phenyl)-4-(4-chloro-2-fluoro-phenyl)-4-cyano-5-(2,2-dimethyl-propyl)-pyrrolidine-2-carboxylic acid trifluoroacetic acid as a brown oil (0.3 g, 100%)

### Example 63

Preparation of intermediate rac-(2R,3S,4R,5S)-1-[2-(tert-butyl-dimethyl-silanyloxy)-ethyl]-3-(3-chloro-2-fluoro-phenyl)-4-(4-chloro-2-fluoro-phenyl)-4-cyano-5-(2,2-dimethyl-propyl)-pyrrolidine-2-carboxylic acid [2-((S)-2,2-dimethyl-[1,3]dioxolan-4-yl)-ethyl]-amide

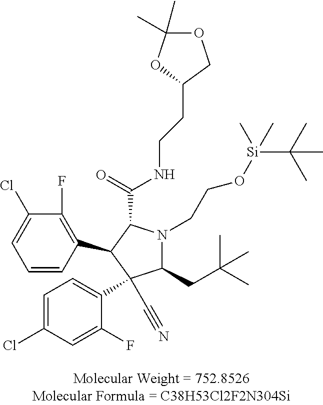

To a mixture of rac-(2R,3S,4R,5S)-1-[2-(tert-butyl-dimethyl-silanyloxy)-ethyl]-3-(3-chloro-2-fluoro-phenyl)-4-(4-chloro-2-fluoro-phenyl)-4-cyano-5-(2,2-dimethyl-propyl)-pyrrolidine-2-carboxylic acid trifluoroacetic acid (0.3 g, 0.48 mmol) in dichloromethane (20 mL) was added 2-((S)-2,2-dimethyl-[1,3]dioxolan-4-yl)-ethylamine (0.23 g, 1.6 mmol), HATU (0.45 g, 1.2 mmol) and iPr₂NEt (0.6 g, 3.8 mmol) respectively. The reaction mixture was stirred at room temperature for 18 h. The mixture was concentrated, and the residue was partitioned between ethyl acetate and water. The organic layer was separated, and aqueous layer was extracted with ethyl acetate. The organic layers were combined, dried over Na₂SO₄, and concentrated. The residue was purified by chromatography (20-40% of EtOAc in Hexanes) to give rac-(2R,3S,4R,5S)-1-[2-(tert-butyl-dimethyl-silanyloxy)-ethyl]-3-(3-chloro-2-fluoro-phenyl)-4-(4-chloro-2-fluoro-phenyl)-4-cyano-5-(2,2-dimethyl-propyl)-pyrrolidine-2-carboxylic acid [2-((S)-2,2-dimethyl-[1,3]dioxolan-4-yl)-ethyl]-amide as a white gum (0.35 g, 96%).

### Example 64

Preparation of intermediate rac-(2R,3S,4R,5S)-3-(3-chloro-2-fluoro-phenyl)-4-(4-chloro-2-fluoro-phenyl)-4-cyano-5-(2,2-dimethyl-propyl)-1-(2-hydroxy-ethyl)-pyrrolidine-2-carboxylic acid [2-((S)-2,2-dimethyl-[1,3]dioxolan-4-yl)-ethyl]-amide

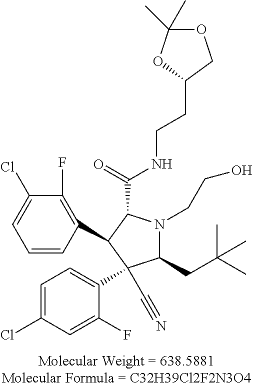

To a solution of rac-(2R,3S,4R,5S)-1-[2-(tert-butyl-dimethyl-silanyloxy)-ethyl]-3-(3-chloro-2-fluoro-phenyl)-4-(4-chloro-2-fluoro-phenyl)-4-cyano-5-(2,2-dimethyl-propyl)-pyrrolidine-2-carboxylic acid [2-((S)-2,2-dimethyl-[1,3]dioxolan-4-yl)-ethyl]-amide (0.35 g, 0.47 mmol) in tetrahydrofuran (20 mL) at 0° C. was added a tetrahydrofuran solution (1 M, Aldrich) of tetrabutylammonium fluoride (1 mL, 1 mmol). The reaction mixture was warmed to room temperature and stirred for 1 h. The mixture was concentrated, and the residue was partitioned between ethyl acetate and water. The organic layer was separated, and aqueous layer was extracted with ethyl acetate. The combined organic layers were washed with brine, dried over MgSO₄, and concentrated. The residue was purified by chromatography (5% MeOH in ethyl acetate) to give rac-(2R,3S,4R,5S)-3-(3-chloro-2-fluoro-phenyl)-4-(4-chloro-2-fluoro-phenyl)-4-cyano-5-(2,2-dimethyl-propyl)-1-(2-hydroxy-ethyl)-pyrrolidine-2-carboxylic acid [2-((S)-2,2-dimethyl-[1,3]dioxolan-4-yl)-ethyl]-amide as a white solid (0.3 g, 100%).

### Example 65

Preparation of intermediate methanesulfonic acid rac-2-[(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-2-(2,2-dimethyl-propyl)-pyrrolidin-1-yl]-ethyl ester

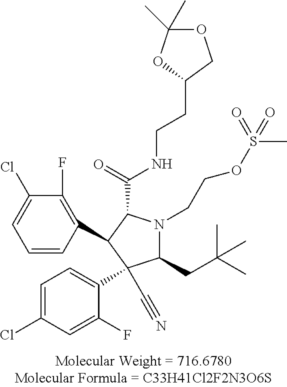

To the solution of rac-(2R,3S,4R,5S)-3-(3-chloro-2-fluoro-phenyl)-4-(4-chloro-2-fluoro-phenyl)-4-cyano-5-(2,2-dimethyl-propyl)-1-(2-hydroxy-ethyl)-pyrrolidine-2-carboxylic acid [2-((S)-2,2-dimethyl-[1,3]dioxolan-4-yl)-ethyl]-amide (0.3 g, 0.47 mmol) and triethylamine (0.14 g, 1.4 mmol) in dichloromethane (20 mL) at 0° C. was added a dichlormethane solution (10 mL) of methanesulfonyl chloride (Aldrich) (80 mg, 0.7 mmol). The reaction mixture was stirred at 0° C. for 1 h. Water was added. Organic layer was separated, the aqueous layer was extracted with dichlormethane. The combined organic layers were washed with diluted aqueous HCl solution, saturated aqueous NaHCO₃ solution, brine, dried over MgSO₄, and concentrated to give methanesulfonic acid rac-2-[(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-2-(2,2-dimethyl-propyl)-pyrrolidin-1-yl]-ethyl ester as a white amorphous (0.33 g, 98%).

### Example 66

Preparation of intermediate rac-(6S,7R,8S,8aR)-8-(3-chloro-2-fluoro-phenyl)-7-(4-chloro-2-fluoro-phenyl)-2-[2-4S)-2,2-dimethyl-[1,3]dioxolan-4-yl)-ethyl]-6-(2,2-dimethyl-propyl)-1-oxo-octahydro-pyrrolo[1,2-a]pyrazine-7-carbonitrile

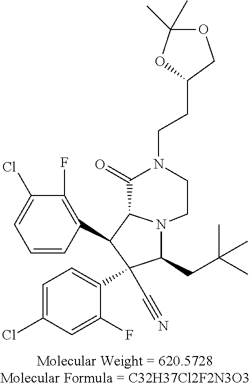

To the solution of methanesulfonic acid rac-2-[(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-2-(2,2-dimethyl-propyl)-pyrrolidin-1-yl]-ethyl ester (0.33 g, 0.46 mmol) in anhydrous dimethylformamide (10 mL) was added Cs₂CO₃ (1 g, 3 mmol). The reaction mixture was heated at room temperature for 2 h. The mixture was partitioned between ethyl acetate and water. The organic layer was separated, and the aqueous layer was extracted with ethyl acetate. The combined organic layers were washed with saturated aqueous NaHCO₃ solution, water, brine, dried over MgSO₄, and concentrated to give rac-(6S,7R,8S,8aR)-8-(3-chloro-2-fluoro-phenyl)-7-(4-chloro-2-fluoro-phenyl)-2-[2-((S)-2,2-dimethyl-[1,3]dioxolan-4-yl)-ethyl]-6-(2,2-dimethyl-propyl)-1-oxo-octahydro-pyrrolo[1,2-a]pyrazine-7-carbonitrile as a white foam (0.29 g, 100%).

### Example 67

### Preparation of rac-(6S,7R,8S,8aR)-8-(3-chloro-2-fluoro-phenyl)-7-(4-chloro-2-fluoro-phenyl)-2-((S)-3,4-dihydroxy-butyl)-6-(2,2-dimethyl-propyl)-1-oxo-octahydro-pyrrolo[1,2-a]pyrazine-7-carbonitrile

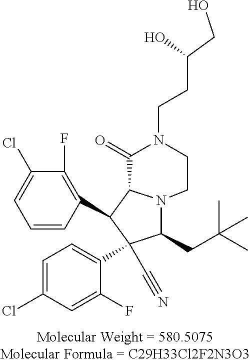

To a solution of give rac-(6S,7R,8S,8aR)-8-(3-chloro-2-fluoro-phenyl)-7-(4-chloro-2-fluoro-phenyl)-2-[2-4S)-2,2-dimethyl-[1,3]dioxolan-4-yl)-ethyl]-6-(2,2-dimethyl-propyl)-1-oxo-octahydro-pyrrolo[1,2-a]pyrazine-7-carbonitrile (0.29 g, 0.47 mol) in tetrahydrofuran (20 mL) was added aqueous HCl solution (1N, 10 mL). The reaction mixture was stirred at room temperature for 2 h, then concentrated. Then the residue was partitioned between ethyl acetate and water. The organic layer was separated, washed with water, aqueous saturated NaHCO₃, brine, dried over MgSO₄, concentrated, dried under reduced pressure to give a white solid (0.26 g, 95%).

HRMS (ES⁺) m/z Calcd for C₂₉H₃₃Cl₂F₂N₃O₃ [(M+H)⁺]: 580.1940. Found: 580.1939.

### Example 68

### Preparation of chiral (6S,7R,8S,8aR)-8-(3-chloro-2-fluoro-phenyl)-7-(4-chloro-2-fluoro-phenyl)-2-((S)-3,4-dihydroxy-butyl)-6-(2,2-dimethyl-propyl)-1-oxo-octahydro-pyrrolo[1,2-a]pyrazine-7-carbonitrile

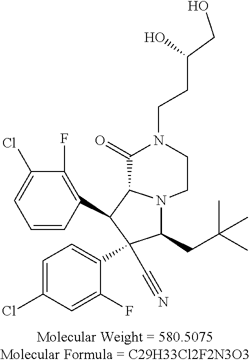

Rac-(6S,7R,8S,8aR)-8-(3-chloro-2-fluoro-phenyl)-7-(4-chloro-2-fluoro-phenyl)-2-((S)-3,4-dihydroxy-butyl)-6-(2,2-dimethyl-propyl)-1-oxo-octahydro-pyrrolo[1,2-a]pyrazine-7-carbonitrile (0.24 g) was separated by chiral SFC chromatography to provide chiral (6S,7R,8S,8aR)-8-(3-chloro-2-fluoro-phenyl)-7-(4-chloro-2-fluoro-phenyl)-2-((S)-3,4-dihydroxy-butyl)-6-(2,2-dimethyl-propyl)-1-oxo-octahydro-pyrrolo[1,2-a]pyrazine-7-carbonitrile as a white solid (0.11 g, 46%) and chiral (6R,7S,8R,8aS)-8-(3-chloro-2-fluoro-phenyl)-7-(4-chloro-2-fluoro-phenyl)-2-((S)-3,4-dihydroxy-butyl)-6-(2,2-dimethyl-propyl)-1-oxo-octahydro-pyrrolo[1,2-a]pyrazine-7-carbonitrile as a white solid (0.12 g, 50%).

### Example 69

Preparation of chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(1R,2R)-2-ethoxycarbonyl-cyclopropyl)-1-oxo-tetrahydro-pyrrolo[1,2-c]imidazol-2-yl]-3-methoxy-benzoic acid methyl ester

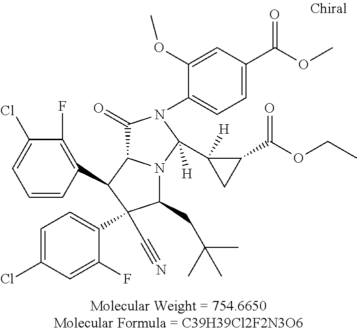

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-3-methoxy-benzoic acid methyl ester (WO 2010031713 A1, 100.0 mg, 0.159 mmol) was combined with AcOH (1.2 mL) then sodium triacetoxyborohydride (Fluka, 300 mg, 8.92 mmol) was added and stirred 15 min. Then ethyl 2-formyl-1-cyclopropanecarboxyalate (107 mg, 0.759 mmol, predominately trans) was added in two portions, 1 h interval. The reaction mixture was stirred an additional 3 hours at room temperature. The reaction mixture was diluted with 0.1 N NaOH and extracted with EtOAc. The organic layer was separated and concentrated under reduced pressure to afford crude product mix. Purification via RP-HPLC (50-95% acetonitrile/water) to afford chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-[((1R,2R)-2-ethoxycarbonyl-cyclopropyl)-1-oxo-tetrahydro-pyrrolo[1,2-c]imidazol-2-yl]-3-methoxy-benzoic acid methyl ester as an off-white solid (18 mg, 15%) as by-product. HRMS (ES⁺) m/z Calcd for C₃₉H₃₉Cl₂N₃O₆F₂ [(M+H)⁺]: 754.2257. Found: 754.2255.

### Example 70

Preparation of chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-((1S,2S)-2-ethoxycarbonyl-cyclopropyl)-1-oxo-tetrahydro-pyrrolo[1,2-c]imidazol-2-yl]-3-methoxy-benzoic acid

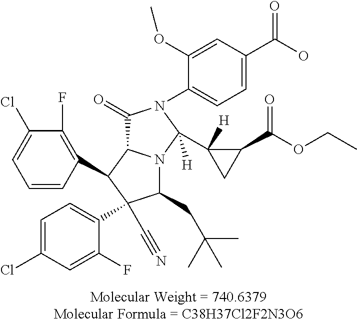

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-3-methoxy-benzoic acid methyl ester (WO 2010031713 A1, 500.0 mg, 0.793 mmol) was combined with AcOH (13.3 mL) then sodium triacetoxyborohydride (Fluka, 500 mg, 2.36 mmol) was added and stirred 15 min. Make sure flask is cool to touch then add ethyl 2-formyl-1-cyclopropanecarboxyalate (500 mg, 3.52 mmol, predominately trans). The reaction mixture was vigorously stirred overnight at room temperature. The reaction mixture was diluted with 0.1 N NaOH and extracted with EtOAc. The organic layer was separated and concentrated under reduced pressure to afford crude products that was taken directly to next step by dissolving in THF (30 mL) followed by addition of 2N LiOH (15 mL) addition. The reaction mixture was stirred at room temperature for 24 hours, then heated to 50° C. for 48 h. Careful work up by separation of aqueous layer, wash with brine, then organic layer separated and concentrated under reduced pressure and purified with RP-HPLC (50-95% acetonitrile/water, 0.05% TFA using JSPHERE ODS-H80, 100×30 mm, s=4 micron; YMCJH08S04-1030WT) to afford chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-((1S,2S)-2-ethoxycarbonyl-cyclopropyl)-1-oxo-tetrahydro-pyrrolo[1,2-c]imidazol-2-yl]-3-methoxy-benzoic acid as an off-white semi-solid (4.7 mg, 0.8%) as byproduct. HRMS (ES⁺) m/z Calcd for C₃₈H₃₇Cl₂F₂N₃O₆ [(M+H)⁺]: 740.2100. Found: 740.2101

### Example 71

Preparation of chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-((1R,2R)-2-ethoxycarbonyl-cyclopropyl)-1-oxo-tetrahydro-pyrrolo[1,2-c]imidazol-2-yl]-3-methoxy-benzoic acid

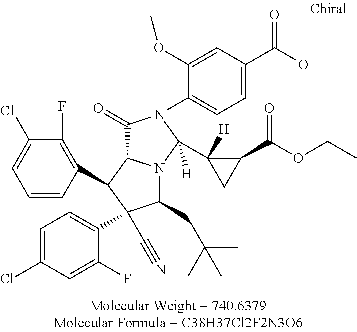

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-3-methoxy-benzoic acid methyl ester (WO 2010031713 A1, 500.0 mg, 0.793 mmol) was combined with AcOH (13.3 mL) then sodium triacetoxyborohydride (Fluka, 500 mg, 2.36 mmol) was added and stirred 15 min. Make sure flask is cool to touch then add ethyl 2-formyl-1-cyclopropanecarboxyalate (500 mg, 3.52 mmol, predominately trans). The reaction mixture was vigorously stirred overnight at room temperature. The reaction mixture was diluted with 0.1 N NaOH and extracted with EtOAc. The organic layer was separated and concentrated under reduced pressure to afford crude products that was taken directly to next step by dissolving in THF (30 mL) followed by addition of 2N LiOH (15 mL) addition. The reaction mixture was stirred at room temperature for 24 hours, then heated to 50° C. for 48 h. Careful work up by separation of aqueous layer, wash with brine, then organic layer separated and concentrated under reduced pressure and purified with RP-HPLC (50-95% acetonitrile/water, 0.05% TFA using JSPHERE ODS-H80, 100×30 mm, s=4 micron; YMCJH08S04-1030WT) to afford chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-((1R,2R)-2-ethoxycarbonyl-cyclopropyl)-1-oxo-tetrahydro-pyrrolo[1,2-c]imidazol-2-yl]-3-methoxy-benzoic acid as an off-white solid (11.4 mg, 1.94%) as the byproduct. HRMS (ES⁺) m/z Calcd for C₃₈H₃₇Cl₂F₂N₃O₆ [(M+H)⁺]: 740.2100. Found: 740.20098.

### Example 72

### In Vitro Activity Assay

The ability of the compounds to inhibit the interaction between p53 and MDM2 proteins was measured by an HTRF (homogeneous time-resolved fluorescence) assay in which recombinant GST-tagged MDM2 binds to a peptide that resembles the MDM2-interacting region of p53. Binding of GST-MDM2 protein and p53-peptide (biotinylated on its N-terminal end) is registered by the FRET (fluorescence resonance energy transfer) between Europium (Eu)-labeled anti-GST antibody and streptavidin-conjugated Allophycocyanin (APC).

Test is performed in black flat-bottom 384-well plates (Costar) in a total volume of 40 uL containing: 90 nM biotinylate peptide, 160 ng/ml GST-MDM2, 20 nM streptavidin-APC (PerkinElmerWallac), 2 nM Eu-labeled anti-GST-antibody (PerkinElmerWallac), 0.2% bovine serum albumin (BSA), 1 mM dithiothreitol (DTT) and 20 mM Tris-borate saline (TBS) buffer as follows: Add 10 uL of GST-MDM2 (640 ng/ml working solution) in reaction buffer to each well. Add 10 uL diluted compounds (1:5 dilution in reaction buffer) to each well, mix by shaking Add 20 uL biotinylated p53 peptide (180 nM working solution) in reaction buffer to each well and mix on shaker. Incubate at 37° C. for 1 h. Add 20 uL streptavidin-APC and Eu-anti-GST antibody mixture (6 nM Eu-anti-GST and 60 nM streptavidin-APC working solution) in TBS buffer with 0.2% BSA, shake at room temperature for 30 minutes and read using a TRF-capable plate reader at

665 and 615 nm (Victor 5, Perkin ElmerWallac), If not specified, the reagents were purchased from Sigma Chemical Co.

Activity data for some of the Example compounds expressed as IC₅₀: bsa: 0.02% are as follows:

| | IC₅₀: bsa: 0.02% |
|---|---|
| Example Number | (IC₅₀, μM) |
| Example 1 | 0.008 |
| Example 2 | 0.008 |
| Example 3 | 0.013 |
| Example 4 | 0.010 |
| Example 6 | 0.013 |
| Example 7 | 0.021 |
| Example 35 | 0.015 |
| Example 41 | 0.009 |

What is claimed:

1. A compound of formula I 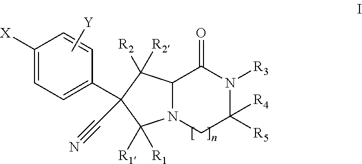
wherein
X is selected from the group consisting of H, F, Cl, Br, I, cyano, nitro, lower alkyl, lower alkynyl and lower alkoxy.
Y is H or F;
n is selected from 0, 1 or 2;
R₁ and R_{1′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₂ and R_{2′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R₄ and R₅ are independently selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or they can together form an oxo group
and a pharmaceutically acceptable salt or ester thereof.

2. The compound of claim 1 having the following stereochemistry 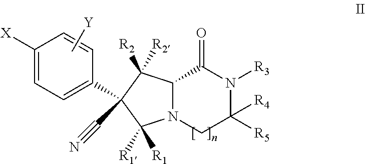
wherein
X is selected from the group consisting of H, F, Cl, Br, I, cyano, nitro, lower alkyl, lower alkynyl and lower alkoxy;
Y is H or F;
n is selected from 0, 1 or 2;
R₁ and R_{1′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₂ and R_{2′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R₄ and R₅ are independently selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or they can together form an oxo group
and the enantiomers thereof and a pharmaceutically acceptable salt or ester thereof.

3. A compound of claim 2 of the formula 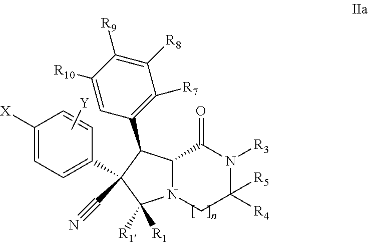
wherein,
R_{2′} is hydrogen;
X is selected from the group consisting of H, F, Cl and Br, I, cyano, nitro, lower alkyl, lower aklynyl and lower alkoxy;
Y is H or F;
n is selected from 0, 1 or 2;
R₈ is selected from the group consisting of F, Cl and Br;
R₇, R₉ and R₁₀ are H or F with the proviso that at least two of R₇, R₉ and R₁₀ are hydrogen;
R₁ and R_{1′} are independently selected from the group consisting of hydrogen, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R₄ and R₅ are independently selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or they can together form an oxo group
and the enantiomers thereof and a pharmaceutically acceptable salt or ester thereof.

4. A compound of claim 3 of the formula 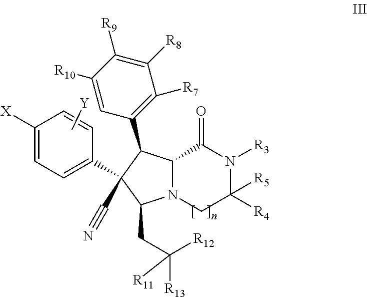
wherein
R_{1′} is hydrogen
X is selected from the group consisting of H, F, Cl and Br, I, cyano, nitro, lower alkyl, lower aklynyl and lower alkoxy;
Y is H or F;
n=0, 1 or 2;
R₈ is selected from the group consisting of F, Cl and Br;
R₇, R₉, R₁₀ are selected from H or F with the proviso that at least two of R₇, R₉ and R₁₀ are hydrogen;
R₁₁, R₁₂ are both methyl, or linked to form a cyclopropyl, cyclobutyl, cyclopentyl or cyclohexyl group;
R₁₃ is (CH₂)ₘ—R₁₄;
m is selected from 0, 1 or 2;
R₁₄ is selected from hydrogen, hydroxyl, lower alkyl, lower alkoxy, lower cycloalkenyl, substituted cycloalkenyl, lower cycloalkyl, substituted cycloalkyl, aryl, substituted aryl, hetereoaryl, substituted heteroaryl, hetereocycle or substituted heterocycle;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R₄ and R₅ are independently selected from H, lower alkyl, substituted lower alkyl, lower cycloalkyl, substituted lower cycloalkyl, lower alkenyl, substituted lower alkenyl, lower cycloalkenyl or substituted lower cycloalkenyl or they can together form an oxo group
and enantiomers and the pharmaceutically acceptable salts and esters thereof.

5. A compound of claim 4 of the formula 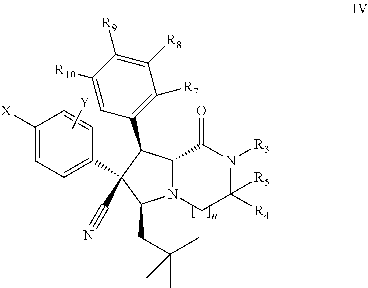
wherein,
X is selected from the group consisting of H, F, Cl and Br, I, cyano, nitro, lower alkyl, lower alkynyl and lower alkoxy;
Y is H or F;
n=0, 1 or 2;
R₈ is selected from the group consisting of F, Cl and Br;
R⁷, R₉, R₁₀ is selected from H or F with the proviso that at least two of R₇, R₉, R₁₀ are hydrogen;
R₃ is selected from the group consisting aryl, substituted aryl, heteroaryl or substituted heteroaryl wherein the substituents are selected from H, carboxyl, amido, hydroxyl, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle;
R₄ and R₅ are independently selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or they can together form an oxo group
and the enantiomers thereof and a pharmaceutically acceptable salt or ester thereof.

6. A compound of claim 5 of the formula 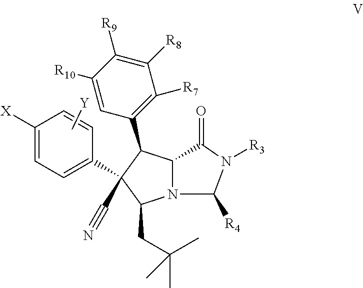
wherein
n=0, R₅=H
X is selected from the group consisting of H, F, Cl and Br, I, cyano, nitro, lower alkyl, lower aklynyl and lower alkoxy;
Y is H or F;
R₈ is selected from the group consisting of F, Cl and Br,
R₇, R₉, R₁₀ is selected from H or F with the proviso that at least two of R₇, R₉, R₁₀ are hydrogen;
R₃ is selected from the group consisting aryl, substituted aryl, heteroaryl or substituted heteroaryl wherein the substituents are selected from H, carboxyl, amido, hydroxyl, cyano, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle;
R₄ is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl;
and the enantiomers thereof and a pharmaceutically acceptable salt or ester thereof.

7. A compound of claim 5 of the formula 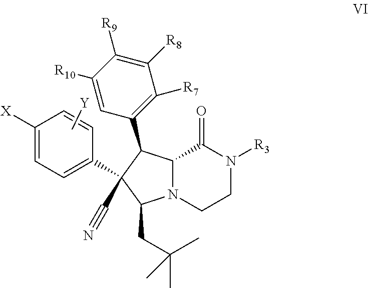
wherein,
X is selected from the group consisting of H, F, Cl, Br, I, cyano, nitro, lower alkyl, lower aklynyl and lower alkoxy;
Y is H or F;
R₈ is selected from the group consisting of F, Cl and Br;
R₇, R₉, R₁₀ is selected from H or F with the proviso that at least two of R₇, R₉, R₁₀ are hydrogen;
R₃ is selected from the group consisting aryl, substituted aryl, heteroaryl or substituted heteroaryl wherein the substituents are selected from H, carboxyl, amido, hydroxyl, cyano, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle;
R₄ and R₅ are H;
n=1
and the enantiomers thereof and a pharmaceutically acceptable salt or ester thereof.

8. A compound of claim 2 selected from the group consisting of
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-methoxy-benzoic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-benzoic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-methoxy-benzoic acid;
[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-acetic acid;
2-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-N,N-bis-(2-methoxy-ethyl)-acetamide;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-hydroxy-benzoic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-trifluoromethyl-benzoic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-fluoro-benzoic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-trifluoromethoxy-benzoic acid;
6R,7S,7aR)-7-(3-chloro-2-fluoro-phenyl)-6-(4-chloro-2-fluoro-phenyl)-5-(2,2-dimethyl-propyl)-2-(3-hydroxy-phenyl)-1-oxo-hexahydro-pyrrolo[1,2-c]imidazole-6-carbonitrile and
(5S,6R,7S,7aR)-7-(3-chloro-2-fluoro-phenyl)-6-(4-chloro-2-fluoro-phenyl)-5-(2,2-dimethyl-propyl)-2-(2-hydroxy-phenyl)-1-oxo-hexahydro-pyrrolo[1,2-c]imidazole-6-carbonitrile.

9. A compound of claim 2 selected from the group consisting of
(5S,6R,7S,7aR)-7-(3-chloro-2-fluoro-phenyl)-6-(4-chloro-2-fluoro-phenyl)-5-(2,2-dimethyl-propyl)-2-(2-fluoro-4-methoxy-phenyl)-1-oxo-hexahydro-pyrrolo[1,2-c]imidazole-6-carbonitrile;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzoic acid methyl ester;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzoic acid methyl ester;
4-[(5S,6R,7S,7aR)-7-(3-chloro-2-fluoro-phenyl)-6-(4-chloro-2 fluoro-phenyl)-6-cyano-5-(2,2-dimethyl-propyl)-1-oxo-tetrahydro-pyrrolo[1,2-c]imidazol-2-yl]-3-methoxy-benzamide;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-3-methoxy-benzoic acid methyl ester;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzamide;
5-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-pyridine-2-carboxylic acid;
4-[(5S,6R,7S,7aR)-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-fluoro-5-methoxy-benzoic acid;
6R,7S,7aR)-7-(3-chloro-2-fluoro-phenyl)-6-(4-chloro-2-fluoro-phenyl)-5-(2,2-dimethyl-propyl)-2-(1-methanesulfonyl-piperidin-4-yl)-1-oxo-hexahydro-pyrrolo[1,2-c]imidazole-6-carbonitrile;
6R,7S,7aR)-2-(1-ccetyl-piperidin-4-yl)-7-(3-chloro-2-fluoro-phenyl)-6-(4-chloro-2-fluoro-phenyl)-5-(2,2-dimethyl-propyl)-1-oxo-hexahydro-pyrrolo[1,2-c]imidazole-6-carbonitrile and
rac-(5S,6R,7R,7aR)-7-(3-chloro-phenyl)-6-(4-chlorophenyl)-5-(2,2-dimethyl-propyl)-2-methyl-1,3-dioxo-hexahydro-pyrrolo[1,2-c]imidazole-6-carbonitrile.

10. A compound of claim 2 selected from the group consisting of
rac-(5S,6R,7R,7aR)-7-(3-chloro-phenyl)-6-(4-chlorophenyl)-5-(2,2-dimethyl-propyl)-2-ethyl-1,3-dioxo-hexahydro-pyrrolo[1,2-c]imidazole-6-carbonitrile;
rac-(5R,6R,7R,7aR)-7-(3-chloro-phenyl)-6-(4-chlorophenyl)-5-(2,2-dimethyl-propyl)-2-ethyl-1,3-dioxo-hexahydro-pyrrolo[1,2-c]imidazole-6-carbonitrile;
rac-3-[(5S,6R,7R,7aR)-7-(3-chloro-phenyl)-6-(4-chlorophenyl)-6-cyano-5-(2,2-dimethyl-propyl)-1,3-dioxo-tetrahydro-pyrrolo[1,2-c]imidazole-2-yl]-propionic acid ethyl ester;
rac-(2R,3R,4R,5S)-3-(3-chloro-phenyl)-4-(4-chloro-phenyl)-4-cyano-5-(2,2-dimethyl-propyl)-1-[(4-hydroxy-butylcarbamoyl)-methyl]-pyrrolidine-2-carboxylic acid tert-butyl ester;
rac-(5S,6R,7R,7aR)-7-(3-chloro-phenyl)-6-(4-chloro-phenyl)-5-(2,2-dimethyl-propyl)-2-(4-hydroxy-butyl)-1,3-dioxo-hexahydro-pyrrolo[1,2-c]imidazole-6-carbonitrile;
rac-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid methyl ester;
rac-4-[(5S,6R,7S,7aR)-7-(3-chloro-2-fluoro-phenyl)-6-(4-chloro-2-fluoro-phenyl)-2-(4-cyano-phenyl)-5-(2,2-dimethyl-propyl)-1,3-dioxo-hexahydro-pyrrolo[1,2-c]imidazole-6-carbonitrile;
rac-4-[(5S,6R,7S,7aR)-7-(3-chloro-2-fluoro-phenyl)-6-(4-chloro-2-fluoro-phenyl)-5-(2,2-dimethyl-propyl)-1,3-dioxo-2-phenyl-hexahydro-pyrrolo[1,2-c]imidazole-6-carbonitrile;
rac-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid;
chiral-4-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid and
rac-3-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid ethyl ester.

11. A compound of claim 2 selected from the group consisting of
rac-3-[(5S,6R,7S,7aR)-7-(3-chloro-2-fluoro-phenyl)-6-(4-chloro-2-fluoro-phenyl)-6-cyano-5-(2,2-dimethyl-propyl)-1,3-dioxo-tetrahydro-pyrrolo[1,2-c]imidazol-2-yl]-benzoic acid;
chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-benzoic acid;
rac-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-benzoic acid;
rac-4-[(3S,5S,6R,7S,7aR)-7-(3-chloro-2-fluoro-phenyl)-6-(4-chloro-2-fluoro-phenyl)-6-cyano-3-cyclohexylmethyl-5-(2,2-dimethyl-propyl)-1-oxo-tetrahydro-pyrrolo[1,2-c]imidazol-2-yl]-benzoic acid;
chiral 4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-benzoic acid;
chiral 4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-2-methoxy-benzoic acid;
rac-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-3-methoxy-benzoic acid;
rac 4-[(3S,5S,6R,7S,7aR)-3-butyl-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2c]imidazol-2-yl]-2-methoxy-benzoic acid;
4-[(3S,5S,6R,7S,7aR)-3-but-3-enyl-7-(3-chloro-2-fluoro-phenyl)-6-(4-chloro-2-fluoro-phenyl)-6-cyano-5-(2,2-dimethyl-propyl)-1-oxo-tetrahydro-pyrrolo[1,2-c]imidazol-2-yl]-2-methoxy-benzoic acid;
rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-3-methoxy-benzoic acid and
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-methyl-1-oxo-tetrahydro-pyrrolo[1,2-c]imidazol-2-yl]-3-methoxy-benzoic acid.

12. A compound of claim 2 selected from the group consisting of
rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isobutyl-1-oxo-tetrahydro-pyrrolo[1,2-c]imidazol-2-yl]-3-methoxy-benzoic acid;
chiral-4-[(3S,5S,6R,7S,7aR)-7-(3-chloro-2-fluoro-phenyl)-6-(4-chloro-2-fluoro-phenyl)-6-cyano-3-cyclopropyl-5-(2,2-dimethyl-propyl)-1-oxo-tetrahydro-pyrrolo[1,2-c]imidazol-2-yl]-3-methoxy-benzoic acid;
chiral-4-[(3S,5S,6R,7S,7aR)-7-(3-chloro-2-fluoro-phenyl)-6-(4-chloro-2-fluoro-phenyl)-6-cyano-3-cyclohexylmethyl-5-(2,2-dimethyl-propyl)-1-oxo-tetrahydro-pyrrolo[1,2-c]imidazol-2-yl]-3-methoxy-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-isopropyl-1-oxo-tetrahydro-pyrrolo[1,2-c]imidazol-2-yl]-3-methoxy-benzoic acid;
chiral-4-[(3S,5S,6R,7S,7aR)-7-(3-chloro-2-fluoro-phenyl)-6-(4-chloro-2-fluoro-phenyl)-6-cyano-3-cyclobutyl-5-(2,2-dimethyl-propyl)-1-oxo-tetrahydro-pyrrolo[1,2-c]imidazol-2-yl]-3-methoxy-benzoic acid;
rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-hydroxymethyl-1-oxo-tetrahydro-pyrrolo[1,2-c]imidazol-2-yl]-benzoic acid;
(2,2-dimethyl-propyl)-3-ethyl-1-oxo-tetrahydro-pyrrolo[1,2c]imidazol-2-yl]-2-methoxy-benzoic acid;
rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-3-methoxy-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-hydroxymethyl-1-oxo-tetrahydro-pyrrolo[1,2-c]imidazol-2-yl]-3-methoxy-benzoic acid;
rac-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-1-oxo-3-(tetrahydro-pyran-4-ylmethyl)-tetrahydro-pyrrolo[1,2-c]imidazol-2-yl]-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-1-oxo-3-(tetrahydro-pyran-4-ylmethyl)-tetrahydro-pyrrolo[1,2-c]imidazol-2-yl]-2-methoxy-benzoic acid;
chiral-4-[(3S,5S,6R,7S,7aR)-7-(3-chloro-2-fluoro-phenyl)-6-(4-chloro-2-fluoro-phenyl)-6-cyano-5-(2,2-dimethyl-propyl)-3-(4-hydroxy-butyl)-1-oxo-tetrahydro-pyrrolo[1,2-c]imidazol-2-yl]-2-methoxy-benzoic acid;
rac-(6S,7R,8S,8aR)-8-(3-chloro-2-fluoro-phenyl)-7-(4-chloro-2-fluoro-phenyl)-2-((S)-3,4-dihydroxy-butyl)-6-(2,2-dimethyl-propyl)-1-oxo-octahydro-pyrrolo[1,2-a]pyrazine-7-carbonitrile and
chiral (6S,7R,8S,8aR)-8-(3-chloro-2-fluoro-phenyl)-7-(4-chloro-2-fluoro-phenyl)-2-((S)-3,4-dihydroxy-butyl)-6-(2,2-dimethyl-propyl)-1-oxo-octahydro-pyrrolo[1,2-a]pyrazine-7-carbonitrile.

13. A pharmaceutical composition comprising a compound of claim 2, or a pharmaceutically acceptable salt or ester thereof, as an active ingredient together with a pharmaceutically acceptable carrier or excipient.
